(19) United States
(12) Patent Application Publication
(10) Pub. No.: US 2014/0341983 A1
(43) Pub. Date: Nov. 20, 2014

(54) SMART™ SOLID ORAL DOSAGE FORMS

(71) Applicants: Donn M. Dennis, Gainesville, FL (US); Richard J. Melker, Gainesville, FL (US)

(72) Inventors: Donn M. Dennis, Gainesville, FL (US); Richard J. Melker, Gainesville, FL (US)

(21) Appl. No.: 14/344,221

(22) PCT Filed: Sep. 14, 2012
(86) PCT No.: PCT/US2012/055627

§ 371 Date: Mar. 11, 2014

Related U.S. Application Data
(60) Provisional application No. 61/534,418, filed on Sep. 14, 2011.

(51) Int. Cl. G01N 33/497; A61K 47/14; A61K 47/10; A61K 47/06

(52) U.S. Cl. CPC G01N 33/497; A61K 47/06; A61K 47/14; A61K 47/10; G01N 2033/4975
USPC 424/451; 424/490; 424/464; 436/56

(57) ABSTRACT

Solid Oral Dosage Forms (SODFs) comprising Self Monitoring and Reporting Therapeutics (SMART™) adherence technology are provided which require no or minimal modification of clinical trial materials (CTMs) or marketed drug while providing tamper resistant (literally foolproof) measurement of adherence that is highly accurate and without altering the chemical, manufacturing, and controls (CMC) of the CTM or marketed drug.

### FIELD OF THE INVENTION

Solid Oral Dosage Forms (SODFs) comprising Self Monitoring and Reporting Therapeutics (SMART™) adherence technology.

### BACKGROUND OF THE INVENTION

### A. Summary of SMART™ Development Program

Xhale, Inc. is developing a technology termed the SMART™ (Self Monitoring and Reporting Therapeutics) adherence system that accurately confirms whether the right person took the right dose of the right drug via the right route at the right time. We call this type of adherence assessment “definitive” because it would be very difficult, if not impossible, for subjects to deceive the system and SMART™ would reliably indicate that the person actually self administered the drug or was administered the medication, for instance, by a caregiver.

The SMART™ adherence system, essentially a personalized medicine tool that provides a significantly better understanding of drug safety and efficacy, is designed to operate in all clinical trial and disease management environments, including the home. It contains two key components: 1) the SMART™ drug, which generates a marker or markers that appears in human breath, termed Exhaled Drug Ingestion Markers (EDIMs), to confirm definitive medication adherence, and 2) the SMART™ device, which accurately measures the EDIMs, provides medication reminder functions, and orchestrates critical adherence information flow between the relevant stakeholders. Typical sensing technologies used to measure EDIMs include but are not limited to mGC-MOS sensors, surface acoustic wave (SAW) sensors, or ion mobility spectroscopy (IMS) sensors. The inventors have broadly taught this art in previous patent applications: Marker detection method and apparatus to monitor drug compliance (filed Apr. 1, 2005; application Ser. No. 11/097,547; U.S. patent application 11/097,647; Pub. No.: US 2005/0233459 A1 by Melker et al), Drug adherence monitoring system-(filed Mar. 7, 2007; U.S. patent application 11/715,197; US 20070224128A1; Pub. No.: US 2007/0224128 A1 by Dennis et al), and Medication Adherence Monitoring System (Provisional application No. 60/891,085, filed on Feb. 22, 2007; U.S. patent application 12/064,673—Filed Feb. 22, 2008; Pub. No.: US 2010/0255598 A1).

In order for pharmaceutical companies to broadly utilize SMART™ adherence for solid oral dosage forms (SODFs) in clinical trials or disease management, novel strategies to package taggants (GRAS flavorants) with clinical trial materials (CTMs) and marketed drugs should meet two criteria: 1) the method of packaging specific GRAS flavorants (taggants) to the medication should provide a tamper resistant (literally foolproof) measurement of adherence that is highly accurate; and 2) the method of packaging the GRAS flavorants (taggants) to SODF-based medications should ideally not alter the chemical, manufacturing, and controls (CMC) of the CTM or marketed drug. The technology described herein provides an invention to accomplish both goals. Although similar formulation architectural approaches (e.g., multi-phase, multi-compartment capsules; capsule in capsule systems; InnerCap Technologies: http://www.innercap.com) exist, they are designed to optimize drug delivery (controlled release; alter absorption and dissolution rates) and are not designed to measure and monitor medication adherence.

### B. Background on SMART™ Adherence

To date, Xhale, Inc. has focused its development efforts on developing the SMART™ adherence system for SODFs, particularly tablet- or capsule-based medications, which are swallowed, enter the stomach, and absorbed in the gastrointestinal tract. In this case, definitive adherence is indicated by the detection of a metabolite of a taggant (GRAS flavorant) as the EDIM. The taggant is packaged together with the final SODF. In this embodiment, our final SMART™ adherence system has successfully employed 1) various formulation strategies that incorporate taggants into the final dosage form without altering the CMC per se of the CTM or marketed drug, and 2) a mGC-MOS as the SMART™ device to measure the EDIMs.

Prior to describing the current invention, a brief review of some key aspects of taggant chemistry outlined in the above referenced patents is provided. Consider a scenario where a patient with a specific disease ingests an active drug, A, for treatment, which is metabolized by enzyme(s) to A1 plus other irrelevant metabolites. In this example, a safe taggant (e.g., GRAS flavorant) without pharmacological activity called T, which may be metabolized to a major metabolite, T1 plus other irrelevant metabolites, is packaged with A. Thus, the two relevant metabolic reactions are: 1: A→A1+others 2: T→T1+others

With regard to measuring a marker(s) that appears in breath, the EDIM(s), which can be measured to verify that A was orally ingested by the patient, we have 4 obvious candidates: 1) A; 2) a major metabolite of A, A1; 3) a taggant, T, which was ingested with the medication containing A; or 4) a metabolite of any taggant (T), T1, which was generated via enzyme metabolism of a taggant (T). The appearance of T1 about 5-10 min later in the breath can be used to document the active drug A (API) was actually ingested. To optimize performance of the adherence system, we have developed a system that will detect at least two markers in the breath within 5-10 min of ingestion to indicate definitive adherence: T and T1 (see section B.1. for details).

### SUMMARY OF THE INVENTION

This patent disclosure provides detailed disclosure for production of novel SODFs which contain markers for definitive medication adherence monitoring. The novel SODFs are useful in a wide range of contexts, including, but not limited to, clinical trial settings, home use settings, hospice, old-age care, or other settings, where it is necessary to definitively confirm that a given patient has taken or been administered a given medication at the correct time and in the correct dosage.

Accordingly, it is an object of this invention to provide novel Solid Oral Dosage Forms (SODFs) with geometries and chemistries that optimize the efficacy of SMART™ (Self Monitoring and Reporting Therapeutics) systems.

Another object of this invention is to provide novel combinations of SMART™ markers.

Another object of this invention is to provide compositions, systems and methodology for application of SMART™ technology to medication adherence monitoring, while requiring minimal modification of the regulatory profile for Active Pharmaceutical Ingredients (APIs).

Other objects and advantages of this invention will be apparent to those of skill in the art from a review of the entire disclosure and the appended claims.

### BRIEF DESCRIPTION OF THE DRAWINGS

FIG. 1: An Illustrative Taggant Formulation for Adherence with Ideal Characteristics. Each component of the taggant (GRAS flavorants) mixture contributes important properties towards the optimal function of SMART™ adherence. All the components are direct food additives and are safe from a toxicology perspective. Specifically, they have favorable permissible daily exposures (PDEs) and acceptable daily intakes (ADIs), which markedly exceed the doses required for SMART™ adherence. For example, relative to the 60 mg dose required in SMART™ adherence, the PDEs (dose that can be taken for remainder of life without regulatory concern) of 2-butanol and 2-pentanone are 300 mg/day and 250 mg/day, respectively. Likewise, the taggant mixture provides the very reliable appearance of EDIMs (e.g., 2-butanone from 2-butanol via ADH, and 2-pentanone directly) to confirm definitive adherence, even under conditions of genetic polymorphisms, environmental effects, and diet. The EDIMs include 2-butanone and 2-pentanone, which are detected in breath using a mGC-MOS breath sensor. The 2-pentanone not only serves as an EDIM for use in combination with 2-butanone, but may well improve absorption of 2-butanol in the stomach and facilitate the conversion of 2-butanol to 2-butanone via ADH. Furthermore, the mixture also has the following advantages: 1) tolerable to subjects (e.g., L-carvone provides a spearmint-like taste), 2) stable to long term storage in hard gel capsules with minimal hydroscopic forces (e.g., hydroxypropyl cellulose (HPC) “ties up” hydrogen bonding of 2-butanol, which in turn reduces its ability to attract water from the hard gel matrix) that would dehydrate the hard gel capsule and reduce its performance, 3) provides acceptable volatility and flammability, and 4) provides suitable viscosity and surface tension for accurately filling large numbers of hard gel capsules during the manufacturing process.

FIG. 2: SMART™ Taggant Packaging Strategies for Tablets

FIG. 3: SMART™ Taggant Packaging Strategies for Capsules

FIG. 4: Properties of Secondary Alcohols that are GRAS Flavorants, Part 1. Shown are 2° alcohols that are listed in the food database as flavorants, and their corresponding ketones, when metabolized via alcohol dehydrogenase (ADH). Note the significant chemical diversity of alcohols and ketones, which could be used in SMART™ adherence to label many doses of a drug or different drugs.

FIG. 5: Properties of Secondary Alcohols that are GRAS Flavorants, Part 2. Shown are 2° alcohols that are listed in the food database as flavorants, and their corresponding ketones, when metabolized via alcohol dehydrogenase (ADH). Note the significant chemical diversity of alcohols and ketones, which could be used in SMART™ adherence to label many doses of a drug or different drugs.

FIG. 6: Most Common Shapes of Solid Oral Dosage Forms (SODFs). The “other” shapes include bullet, clover, double circle, freeform (e.g., apple), gear, semi-circle, tear and trapezoid. Names for the 3, 5, 6, 7, and 8 sided forms are triangle, pentagon, hexagon, heptagon, and octagon, respectively.

FIG. 7: Licap® Capsules by Capsugel, Size 000 to 0

FIG. 8: Licap® Capsules by Capsugel, Size 1 to 4

FIG. 9: Coni-Snap® Capsules by Capsugel, Size 000 to 0

FIG. 10: Coni-Snap® Capsules by Capsugel, Size lel to 5

FIG. 11: Double Blind® (DB) Capsules by Capsugel

FIG. 12: Soft Gelatin Products by Capsugel. As shown softgels come in a wide variety of shapes (round, oblong, and oval), colors, sizes (0.75 mm to 30 mm), and volumes (0.046 ml to 2.53 ml). It should be noted that Capsugel can easily customize softgels into any size or shape. In terms of utility in SMART™ adherence, the preferred embodiment would be to use the softgels to contain the ideal taggant mixtures; the softgels would in turn be placed in standard capsules, including but not limited to Licaps, Coni-snaps, DB caps, VCaps, etc.

FIG. 13: Oral Vanillin Detection using Direct Breath Mass Spec (LCMS) Analysis

FIG. 14: Oral Vanillin Detection using Direct Breath LCMS Analysis. After 5-10 sec of the vanillin being placed on the tongue, the subject exhaled into the LCMS and the response is depicted.

FIG. 15: Time-Dependent Decay of Vanillin in Human Breath. Using the LCMS response to gas phase vanillin, the flavorant persists in human breath for 2 min following placement of 30 μg vanillin in 10 μL neat ethanol. Shown is the LCMS response to 4 separate breaths.

FIG. 16. Photographs of SAW Devices Used in the Studies.

FIG. 17. Comparison between unprocessed SAW detector output (A) and baseline-subtracted output (B) for a series of methyl salicylate standards.

FIG. 18. Baseline-subtracted SAW response to standard of 100 ng D-limonene and 30 ng methyl salicylate injected on SAW device 1.

FIG. 19. Comparison of SAW sensitivity for methyl salicylate and D-limonene.

FIG. 20. Standard curves for D-limonene and methyl salicylate obtained on SAW device 1. 250 samples were analyzed by the device between the experiments conducted starting at time t1 and t2, where t2 is 24 days later than t1.

FIG. 21. Average peak height values for 100 ng D-limonene/30 ng methyl salicylate check standards run on the four SAW devices during the clinical study. Error bars are equal to ±1 standard deviation.

FIG. 22. Mass of flavorant exhaled at various times after the sublingual administration of a single formulation containing 300 μg of methyl salicylate plus 300 μg D-limonene. Values displayed are the average of four participants, and error bars equal±1 standard deviation.

FIG. 23. Mass of flavorant exhaled as a function of dose for D-limonene and methyl salicylate obtained 5 seconds after sublingual administration.

FIG. 24. Reproducibility of exhaled flavorant mass following replicate doses of D-limonene and methyl salicylate. Bars show the average value of three replicates, and error bars are equal to ±1 standard deviation.

FIG. 25. Average exhaled mass of D-limonene and methyl salicylate following sublingual administration of 1) 30 mg SL powder with 300 μg D-limonene and 30 μg methyl salicylate (blue bar), 2) 30 mg SL powder with 100 μg D-limonene and 30 μg methyl salicylate (red bar) and 3) 20 μL of ethanol containing 100 μg D-limonene and 30 μg of methyl salicylate (green bar). Error bars equal±1 standard deviation.

FIG. 26. Average mass of D-limonene recovered from 30 mg aliquots of bulk placebo SL powder which had been formulated to contain 200 ng D-limonene per 30 mg of matrix. Bars show average values for three replicate samples taken 0, 4, and 8 hours after preparation of the powder along with the fraction of D-limonene recovered. Error bars equal±1 standard deviation.

FIG. 27. SAW output and interpretation of breath samples obtained during visit 2 for participant SAWO09.

FIG. 28. Observed peak heights for D-limonene and methyl salicylate in breath samples collected during the clinical study in Aim 2.

FIG. 29. Exhaled D-limonene mass measured after administration of formulation 5 in Aim 2.

FIG. 30. The lowest SAW responses for D-limonene and methyl salicylate measured during the Aim 2 clinical trial were observed in subject SAWO10 during one of the study visits. Formulations remain easily distinguishable.

FIG. 31. Total Ion Chromatograph (TIC) of baseline breath sample and breath samples collected after administration of the FONA powders.

FIG. 32. High resolution API mass spectra of methyl salicylate (A) and the breath sample following administration of the wintergreen powder (B).

FIG. 33. High resolution selected ion (SI) chromatograms of the baseline breath samples and breath samples collected after FONA powder administration.

FIG. 34. Concentration of methyl salicylate in the FONA powders.

FIG. 35. GC/MS Analysis of ALAVERT Fresh Mint Tablet (300 mg Tablet containing 10 mg Loratadine).

FIG. 36. GC/MS Analysis of ALAVERT Citrus Blast Tablet (300 mg tablet containing 10 mg Loratadine).

FIG. 37. GC/MS Analysis of Wintergreen Flavor (FONA FIG. 38. SAW Reference Standards—100 ng of limonene and 30 ng methyl salicylate injected directly into the device.

FIG. 39. SAW Reference Standards.

FIG. 40. Alayert™ Fresh Mint ODT.

FIG. 41. Alavert™ Citrus Burst ODT.

FIG. 42. FONA Wintergreen Powder.

### DETAILED DISCLOSURE OF THE PREFERRED EMBODIMENTS OF THE INVENTION

### Definitions

Throughout this disclosure, reference is made to SMART™ markers, taggants, EDIMs. It will be appreciated that the marker, taggant or EDIM is preferably a compound which is Generally Regarded/Recognized as Safe, or is a metabolite of such a compound (i.e. it is a GRAS compound, as defined, for example, at: http://www.fda.gov/Food/FoodIngredientsPackaging/GenerallyRecognizedasSa feGRAS/default.htm, which states, in relevant part: ““GRAS” is an acronym for the phrase Generally Recognized As Safe. Under sections 201(s) and 409 of the Federal Food, Drug, and Cosmetic Act (the Act), any substance that is intentionally added to food is a food additive, that is subject to premarket review and approval by FDA, unless the substance is generally recognized, among qualified experts, as having been adequately shown to be safe under the conditions of its intended use, or unless the use of the substance is otherwise excluded from the definition of a food additive.

Under sections 201(s) and 409 of the Act, and FDA's implementing regulations in 21 CFR 170.3 and 21 CFR 170.30, the use of a food substance may be GRAS either through scientific procedures or, for a substance used in food before 1958, through experience based on common use in food.

Under 21 CFR 170.30(b), general recognition of safety through scientific procedures requires the same quantity and quality of scientific evidence as is required to obtain approval of the substance as a food additive and ordinarily is based upon published studies, which may be corroborated by unpublished studies and other data and information.

Under 21 CFR 170.30(c) and 170.3(f), general recognition of safety through experience based on common use in foods requires a substantial history of consumption for food use by a significant number of consumers.”

While, indeed, it is preferred for the SMART™ taggant, marker, EDIM to be or be derived from a GRAS compound, it should be understood that non-GRAS compounds may be utilized in the SODFs as described herein, without departing from the heart of the present invention. Accordingly, use of non-toxic volatile compounds that are not designated as GRAS is not excluded from the present invention, provided such compounds otherwise meet the criteria for such compounds to be used as SMART™ taggants, markers, EDIMs, as set forth herein below.

By the term “subject” it should be understood that vertebrates, and in particulare mammals, and in particular primates (including humans), cats, dogs, livestock (sheep, pigs, cows and the like), rodents, and the like are intended.

It will be appreciated that while specific examples are provided herein, such examples are not intended to be limiting, and equivalents of the specific examples are likewise to be considered as coming within the scope of the invention as herein disclosed and claimed.

### B.1. Example of Ideal Taggant System

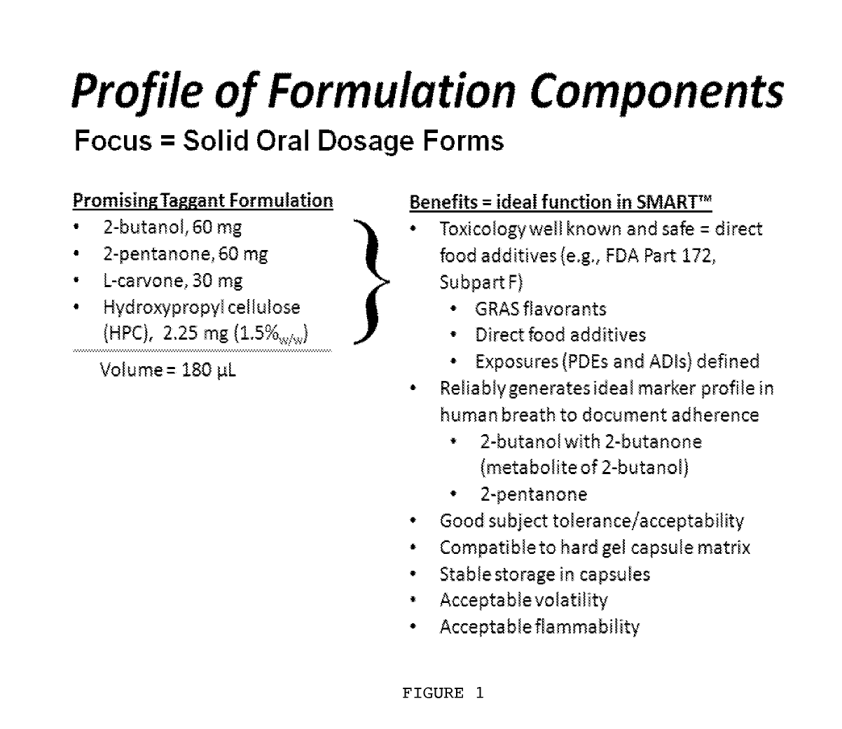

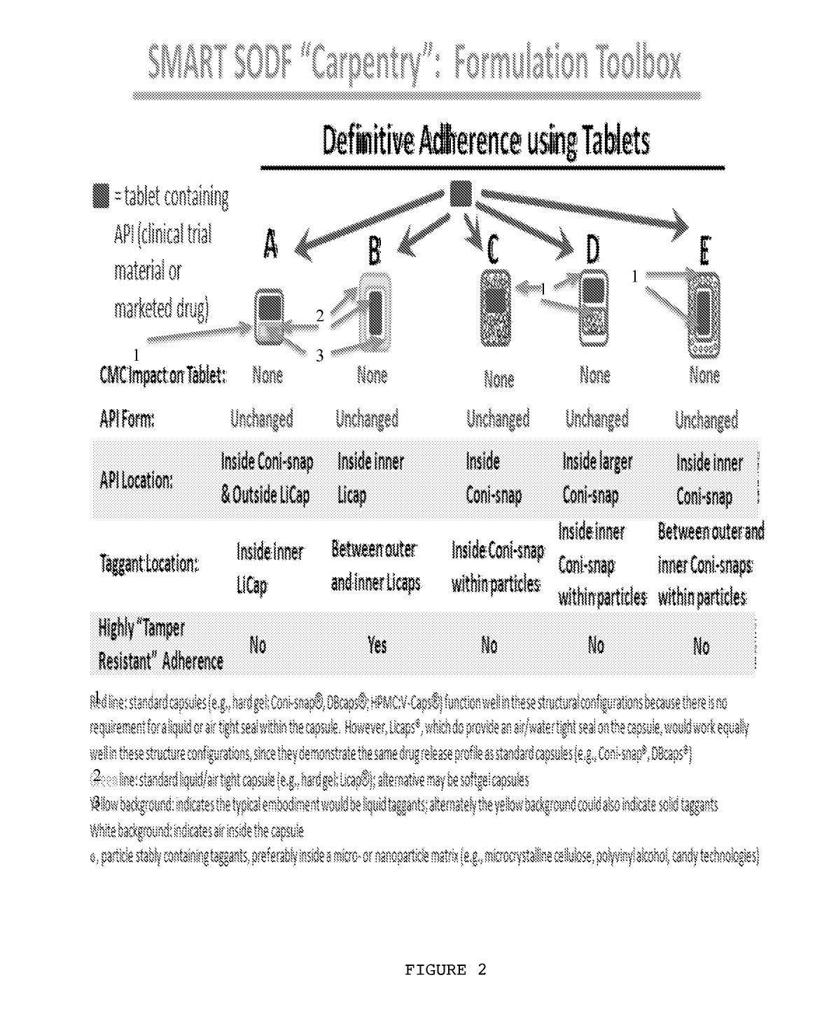

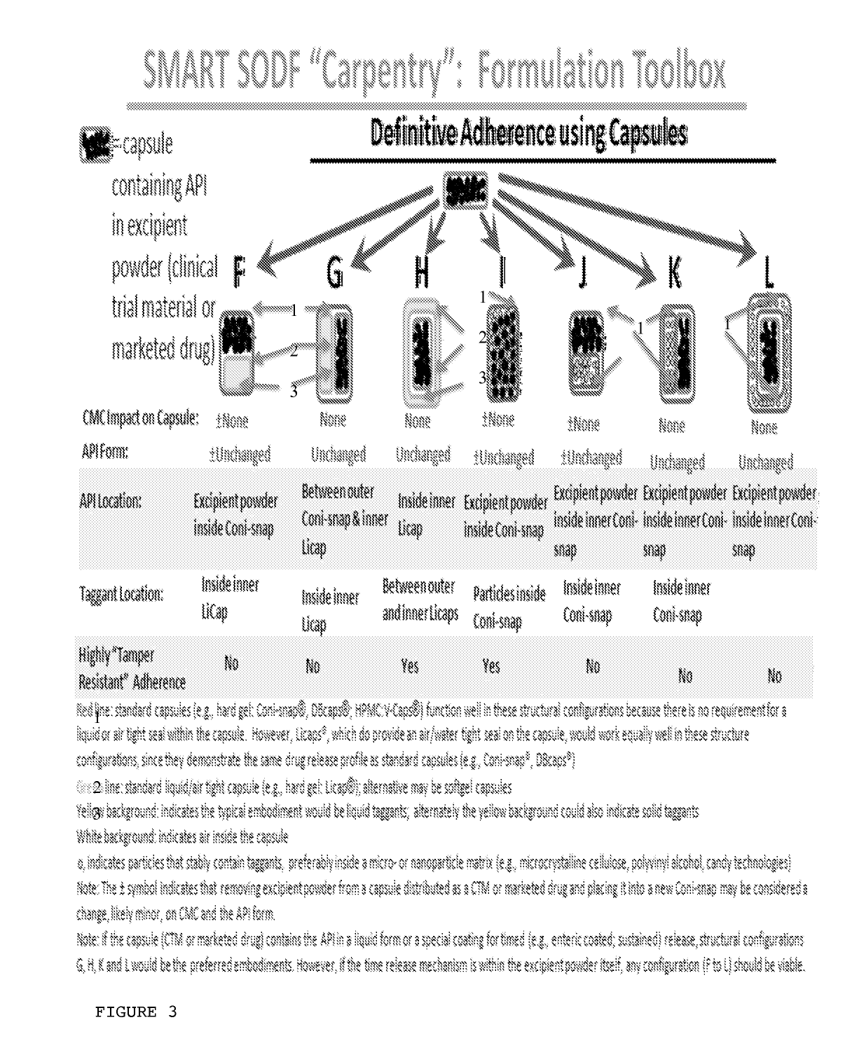

To maximize performance of the SMART™ adherence system, we developed an optimized taggant system (FIG. 1) that contains the following complementary components: 2-butanol (60 mg), 2-pentanone (60 mg), L-carvone (30 mg), and hydroxypropyl cellulose (2.25 mg). This represents one example, but others can be readily created for SMART™ adherence applications. Each component contributes to the overall function of the SMART™ adherence system. Specifically, both type and dose of each component, appears to contribute to optimal SMART™ adherence function. In this configuration, the taggant mixture, when packaged in various structural configurations to a tablet-based (FIG. 2) or to a capsule-based (FIG. 3) SODF type medication, reliably generates at least two complementary EDIMs: 1) T1, namely a ketone generated from a 2° alcohol (i.e., 2-butanone generated from 2-butanol), and 2) T, namely a ketone (i.e., 2-pentanone). In some individuals, 2-butanol can also be detected in breath by the mGC-MOS sensor. In various preclinical and clinical studies, the taggant mixture (FIG. 1) appears to possess the following key characteristics of the ideal SMART™ taggant:

1. It should generate at least one of the EDIMs after in vivo metabolism of the taggant, preferably a GRAS flavorant.
2. It should produce an EDIM(s) that is distinct from compounds that typically appear in the breath as a result of metabolism, diet or disease.
3. Its EDIM(s) should appear in breath within 5-10 min of ingestion of the SMART™ medication.
4. Its EDIM(s) should persist in breath at a detectable concentration for at least 15 min, ideally 1-2 hours, but less than 6 hours for a single daily dose smart medication.
5. The generation of EDIM(s), if the result of metabolism, should occur in the setting of 1^{st} order kinetics.
6. The generation of EDIM(s) should not be significantly affected by genetic polymorphisms or disease in the participating enzymes, and/or the presence of other compounds (e.g., drug-induced inhibition of metabolism).
7. It should be a GRAS compound (e.g., flavorant) to minimize FDA regulatory hurdles.
8. It and its metabolites should have no inherent pharmacological or toxicological properties at the levels needed for medication adherence monitoring.
9. It should not alter the pharmacokinetics (PK) of the API (bioequivalent).
10. It should be inexpensive, readily available and formulated in a manner so that it cannot be readily separated from the active drug, in order to prevent deceptive adherence behavior.
11. The taggant(s) should be packaged with the CTM or marketed drug in a manner that does not alter the CMC of the CTM or marketed drug.
12. The physicochemical properties of the taggant(s) should allow them to be packaged with capsules (e.g., Licaps) in a manner that provides acceptable long term (greater than or equal to 6 months shelf life) stability in capsules, which includes acceptable volatility and flammability and no impact on the viability of the hard gel capsule matrix.
13. The taggant mixture, when given in amounts that will reliably generate adequate EDIMs to document medication adherence, should be readily tolerated by the subject (e.g., taste acceptable, etc.).
14. Physicochemical properties of taggant mixture must allow for mass manufacture (filling) capabilities (e.g., suitable viscosity and surface tension of taggant mixture allows for large scale accurate filling of taggant mixture into Licap capsules).

### B.2. Advantages of Using 2-Butanol as a Key Taggant (GRAS Flavorant) in SMART™ adherence

We found that 2° alcohols, as opposed to 1° alcohols, are superior as taggants for definitive adherence. Specifically, 2° and 1° alcohols, when metabolized by alcohol dehydrogenase (ADH), yield ketones and aldehydes, respectively. Ketones are superior breath markers because they persist longer in breath and generally do not have much, if any, background interference with the exception of acetone. Acetone is a product of carbohydrate metabolism, and is present in human breath at concentrations of 293-870 ppb (Diskin A M et al: Physiol Meas 24:107, 2003). In contrast, aldehydes that are produced from 1° alcohols, do not provide a reliable breath marker for definitive adherence, due to efficient scavenging by aldehyde dehydrogenase (ALDH) and their subsequent conversion to acids.

In addition, from a performance perspective, the breath marker must be reliably generated from metabolism of the taggant by the enzyme. Thus, factors that reduce the function of ADH, will seriously impact system performance! For example, factors such as genetic polymorphisms in different ethnic groups and the presence of ethanol can markedly reduce the ability of ADH to metabolize 1° alcohols. In contrast, the ADH isoforms that degrade 2° alcohols and generate ketones are not affected by these factors, which is a major advantage also favoring the use of 2° alcohols in definitive adherence. From a safety perspective, it is important to note that the permissible daily exposure (PDE) of 2-butanol and 2-pentanone is 300 and 250 mg orally per day, respectively, so the doses (e.g., 60 mg) used in SMART™ adherence are well below any limit of regulatory concern. To put the PDE in perspective in terms of safety margins used in the calculations, the PDE set by the FDA for ethanol is 166.7 mg orally per day, whereas a typical mixed drink (≈1 oz of ethanol) contains 28,300 mg of ethanol.

Another favorable feature of using 2° alcohols with SMART™ adherence is that approximately 20-30% of orally ingested smaller molecular weight alcohols will be absorbed directly through the gastric wall. This feature is highly desirable, because the breath marker (EDIM) appearance is not dependent on small intestinal (e.g., duodenal, jejunal) absorption, which in turn is highly dependent on the extremely variable process of gastric emptying, and will allow early appearance of EDIMs. Factors that commonly alter gastric emptying, include but are not limited to food type, food amount, stress, drugs, and disease (e.g., diabetes) etc can all affect gastric emptying.

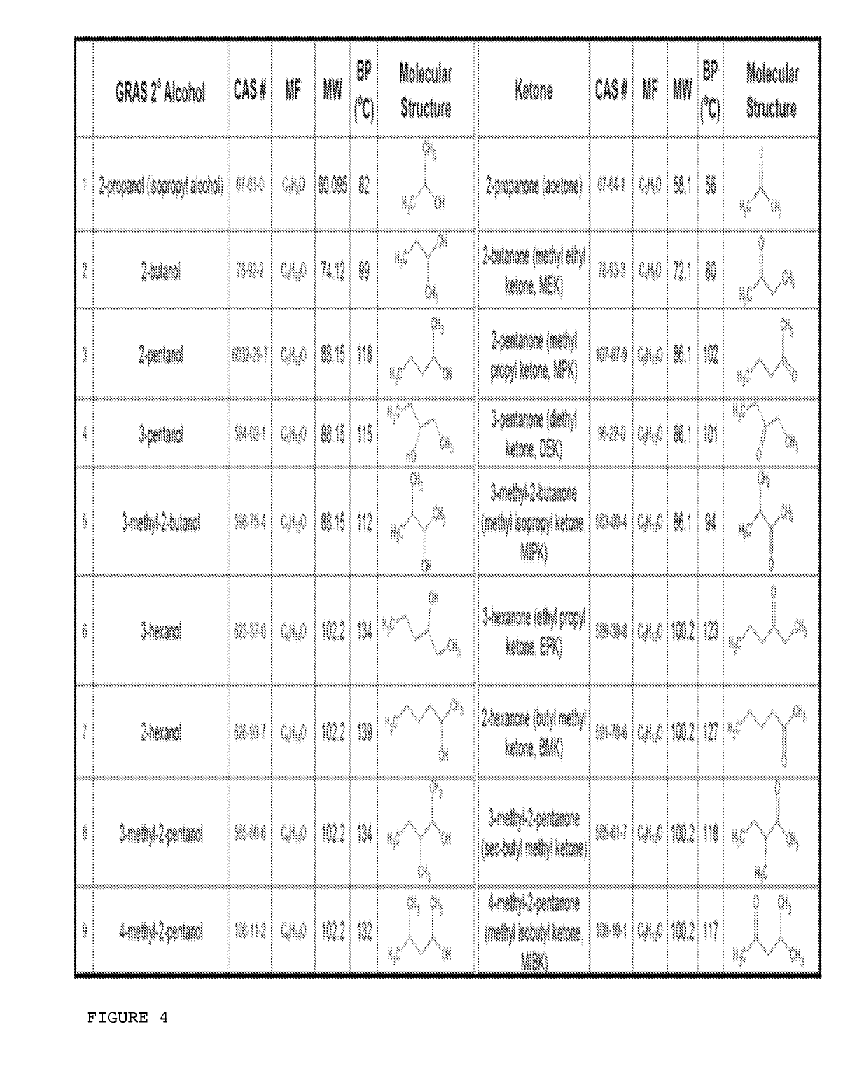

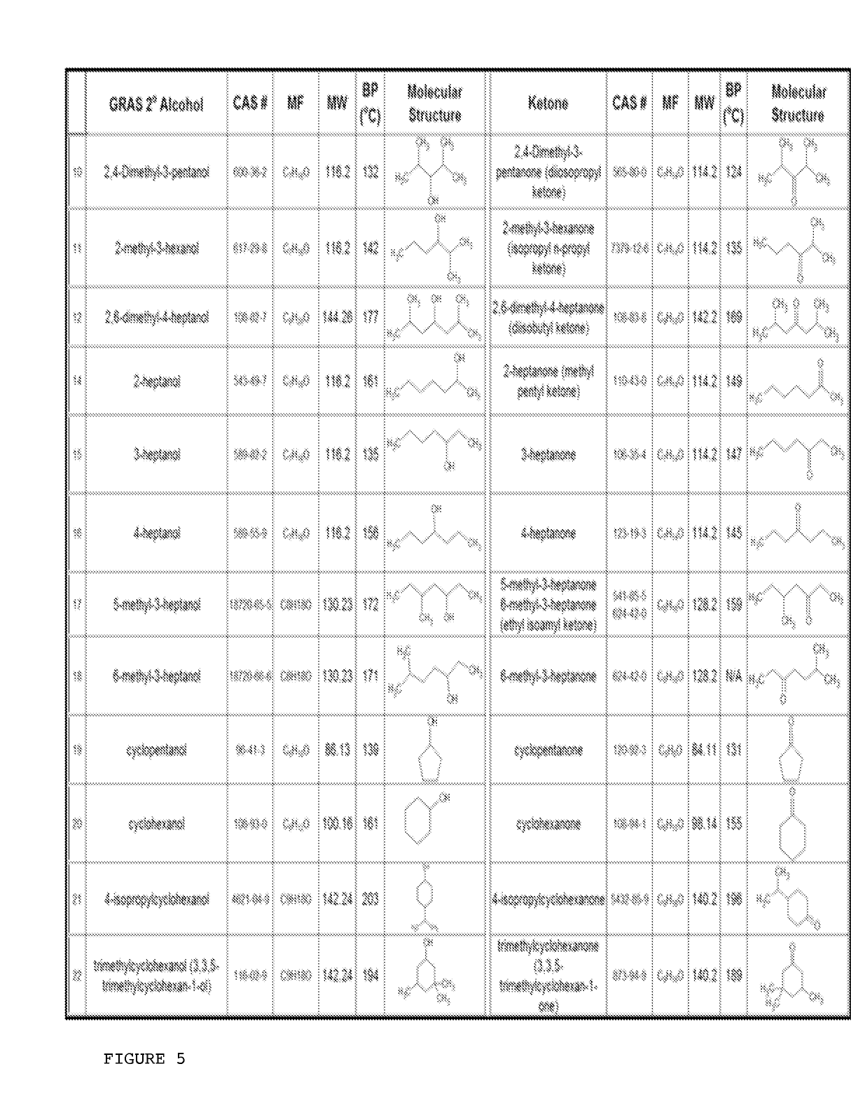

Another major advantage of using 2° alcohols with SMART™ is that the GRAS databases contain a great diversity of 2° alcohols, namely at least 22 of them (FIGS. 4 and 5). Each of these 2° alcohols will individually liberate unique ketones that can be sensed by the mGC-MOS, providing great diversity of tagging different medication forms in the definitive adherence arena. In other words, there is plenty of flexibility with safe chemicals (e.g., GRAS flavorants) to label many different drugs and/or different doses of a given drug with definitive adherence capabilities.

### B.3. ADH Variability Due to Genetic and Environmental Factors Does Not Affect SMART™

Genetic variability in enzyme function does not affect the generation of the ketone, 2-butanone from the 2° alcohol, 2-butanol. The human metabolism of alcohols and related metabolites occur at different rates (e.g., ethnic-based), due to genetic polymorphisms of ADH and ALDH.

ADH¹⁻⁴: Genetic polymorphisms occur at the ADH2 and ADH3 gene loci, which can significantly alter enzymatic function in ADH isozymes containing the β (β₁ and β₂) and γ (γ₁ and γ₂) subunits, respectively. In 85% of eastern Asian populations β₂ is the predominant allele, whereas in 90% of Caucasians β₁ is the major allele. Because β₂β₂ ADH exhibits a higher rate of enzymatic activity relative to other common forms, eastern Asian populations can rapidly convert ethanol to acetaldehyde. These types of ADH genetic issues will not confound our taggant chemistry, because 2° alcohols (e.g., 2-butanol) are preferentially and efficiently metabolized to their ketones by aa ADH, which is not subject to genetic polymorphisms.⁵⁻⁷ These ADH systems remain present and functional with minimal changes in Michaelis and Menten K_{M} even in patients with alcoholic cirrhosis.⁸
ALDH⁹⁻¹⁰: An important enzyme that degrades acetaldehyde formed from ethanol is a microsomal aldehyde dehydrogenase (ALDH) 2 (ALDH2), which has a high affinity for acetaldehyde and rapidly converts it to acetic acid. Because 40-45% of eastern Asians possess inactive ALDH2 due to a point mutation (mutant allele, ALDH2*2), they frequently cannot metabolize acetaldehyde. The ALDH2*2 found in eastern Asians (or even disulfuram-treated subjects) would not alter chemical performance, because the ketone formed from 2-butanol is not degraded by this enzyme and it is mainly lung excreted. In summary we selected a 2° alcohol (e.g., 2-butanol) as a taggant for 2 major reasons: 1) relative to ethanol (and other 1° alcohols), these molecular entities are efficiently (Vₘₐₓ/Kₘ 50-110× greater) and very preferentially (higher binding affinity: K_{M} 0.009-0.025× lower) metabolized by distinct ADH isozymes (e.g., aa ADH)²; this finding also indicates that blood ethanol should not interfere with the generation of the 2-butanone (exhaled marker) when using 2-butanol as a taggant in DAS, and 2) the Class I ADH isozyme that is selective for 2° alcohols (e.g., αα ADH) is not subject to genetic variability.⁵⁻⁷

### Section B.3 References

1. Lieber CS: Alcohol: Its Metabolism and Interactions with Nutrients. Annu Rev Nutr 2000; 20:395-430.
2. Stone Cl, Ting-Kai Li, Bosron WF: Stereospecific oxidation of secondary alcohols by human alcohols dehydrogenases, J Biol Chem, 1989; 264(19):11112-11116. PMID: 2738060.
3. Yin S J, Bosron W F, Magnes L J, Li T K: Human liver alcohol dehydrogenase: purification and kinetic characterization of the beta 2 beta 2, beta 2 beta 1, alpha beta 2, and beta 2 gamma 1 “Oriental” isoenzymes. Biochemistry. 1984; 23(24):5847-5853.
4. Duester G, Smith M, Bilanchone V, Hatfield G W: Molecular analysis of the human class I alcohol dehydrogenase gene family and nucleotide sequence of the gene encoding the beta subunit. J Biol Chem. 1986; 261(5):2027-2033.
5. Reddy, B. M., Reddy, A. N. S., Nagaraja, T., Bhaskar, K. S., Thangaraj, K., Reddy, A. G., Singh, L. (2006). Single Nucleotide Polymorphisms of the Alcohol Dehydrogenase Genes among the 28 Caste and Tribal Populations of India. Int J Hum Genet, 6(4): 309-316.
6. Bosron W. F., Magnes L. J., Li T. K. (1983). Human liver alcohol dehydrogenase: ADH Indianapolis results from genetic polymorphism at the ADH2 gene locus. Biochem Genet. 21:735-744. PMID: 6354175.
7. Edenberg H. J., Bosron W. F. (1997) Alcohol Dehydrogenases, pp 119-131. In: FP Guengerich (Ed). Comprehensive Toxicology. Vol 3. Biotranformation. New Yrok, Pergamon Press.
8. Dam, G., Sorensen, M., Munk, O. L., Keiding, S. (2009). Hepatic ethanol elimination kinetics in patients with cirrhosis. Scand. J. Gastroenterol. 44:867-871. PMID: 19404864.
9. Wall T L, Peterson C M, Peterson K P, Johnson M L, Thomasson H R, Cole M, Ehlers C L: Alcohol metabolism in Asian-American men with genetic polymorphisms of aldehyde dehydrogenase, Ann Intern Med. 1997; 127(5):376-379.
10. Garver E, Tu Gc, Cao Q N, Aini M, Zhou F, Israel Y: Eliciting the low-activity aldehyde dehydrogenase Asian phenotype by an antisense mechanism results in an aversion to ethanol. J Exp Med. 2001; 194(5):571-580.

### B.4. Food Intake does not Reduce the Accuracy of SMART™ Adherence or Impair Function

Although many of these GRAS compounds (including butanol and butanone) are contained within food, the amounts per servings are very low. In order to cause any interference these items will have to be ingested in inconsumably large quantities. To confirm this, we investigated the effect of foods, known to naturally contain the largest quantities of the taggant (e.g., 2-butanol—tomato and black tea) and its volatile metabolite (e.g., 2-butanone—cheddar cheese and yogurt), on system performance. We studied the concentration-time relationships of 2-butanol and 2-butanone in the breath following the ingestion of large quantities of 4 foods: 1) cheddar cheese, 2) yogurt, 3) black tea, and 4) beefsteak tomatoes. To assess the interference produced by food, 6 subjects rapidly consumed (<5 min) two servings (≈16 fl. Oz) of yogurt or 2 servings (≈60 g) of cheddar cheese and breath samples were collected for 60 min after ingestion. Maximum 2-butanone (C_{Max}) values for yogurt and cheddar cheese were 18±4 ng/L (≈6 ppb) at 0 min and 14±3 ng/L (≈5 ppb) at a Tₘₐₓ of 0 min, respectively, and were non-significant. Similar data was noted following black tea (n=8) or tomato (n=8) consumption. For comparison, 2-butanone Cₘₐₓ values (n=7 subjects) after ingestion of 40 mg of 2-butanol taggant was 1620±260 (≈549 ppb) at a Tmax of 6.7±0.6 min (95% CL=5.3-8.1 min), which represents a 100× separation between 2-butanone Cₘₐₓ values for taggant vs food. Thus, the system will not be affected by ingestion of even large quantities of food naturally containing the highest levels of 2-butanol and 2-butanone. Similarly, the effect of fatty meals (e.g., egg McMuffin and Jimmy Dean's muffin) as well as the ingestion of a large meal “heavy” in carbohydrates (e.g., foot long Subway Italian sub consumed over 15 min), despite reducing the concentration-time relationships (area under the curve, AUC) of the EDIMs (e.g., 2-butanone and 2-pentanone), did not prevent accurate determinations of medication adherence (data not shown).

### C. SMART™ Carpentry

The invention disclosed in this patent filing provides at least two advances to the art by disclosing: 1) a prototypical optimized taggant mixture that provides optimal function in SMART™ adherence, and 2) how the optimized taggant mixture can be packaged with widely used SODFs in various complementary structural configurations that cause no CMC changes to the CTM or marketed drug per se.

FIG. 2 and FIG. 3 depict how tablet-based and capsule-based SODF medications, respectively, can be packaged in this manner.

### C.1. SMART™ Carpentry for Tablet-based SODFs

Shown in FIG. 2 are five structural formulation configurations (A, B. C, D, and E) whereby tablets can be converted to SMART™ (self report adherence) versions of the drug using complementary strategies. Specifically, the taggants (GRAS flavorants) are packaged in various ways that do not require the CMC per se of the CTM or marketed drug to be changed, but still provide for accurate assessments of medication adherence.

### C.2. SMART™ Carpentry for Capsule-Based SODFs

Shown in FIG. 3 are seven structural formulation configurations (F, G, H, I, J, K, and L) whereby capsules can be converted to SMART™ (self report adherence) versions of the drug using complementary strategies. Specifically, the taggants (GRAS flavorants) are packaged in various ways that do not require the CMC per se of the CTM or marketed drug to be changed, but still provide for accurate assessments of medication adherence.

### C.3. Specific Embodiments of SMAR™ Carpentry

Virtually any drug, including those in hard tablets or capsules, can be adapted to be detectable in breath by incorporating SMART™ taggants in a way that doesn't alter the pharmacokinetic (PK)/pharmacodynamic (PD) profile of the active pharmaceutical ingredient (API).

Additionally, several hard tablets and/or capsules can be packaged into a single SMART™ drug (e.g. a “Life Style” medication containing a statin, a blood pressure medication and aspirin). This is particularly advantageous is some subpopulations were cognitive function is impaired and adherence to multiple medications must be monitored on a daily (or regular) basis.

C.3.1. Shape of SODF Products that can be Packaged for Definitive Adherence Using SMART™

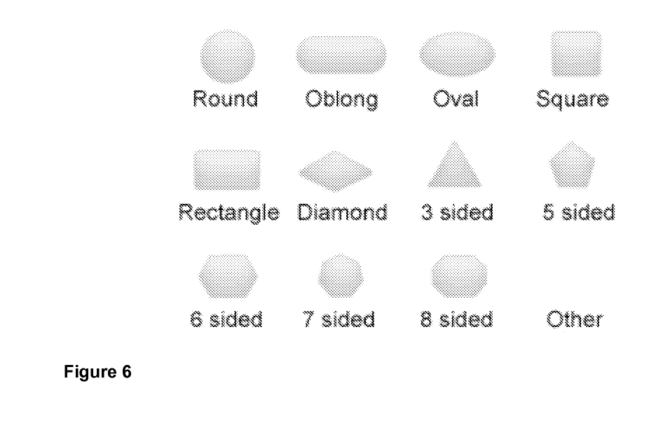

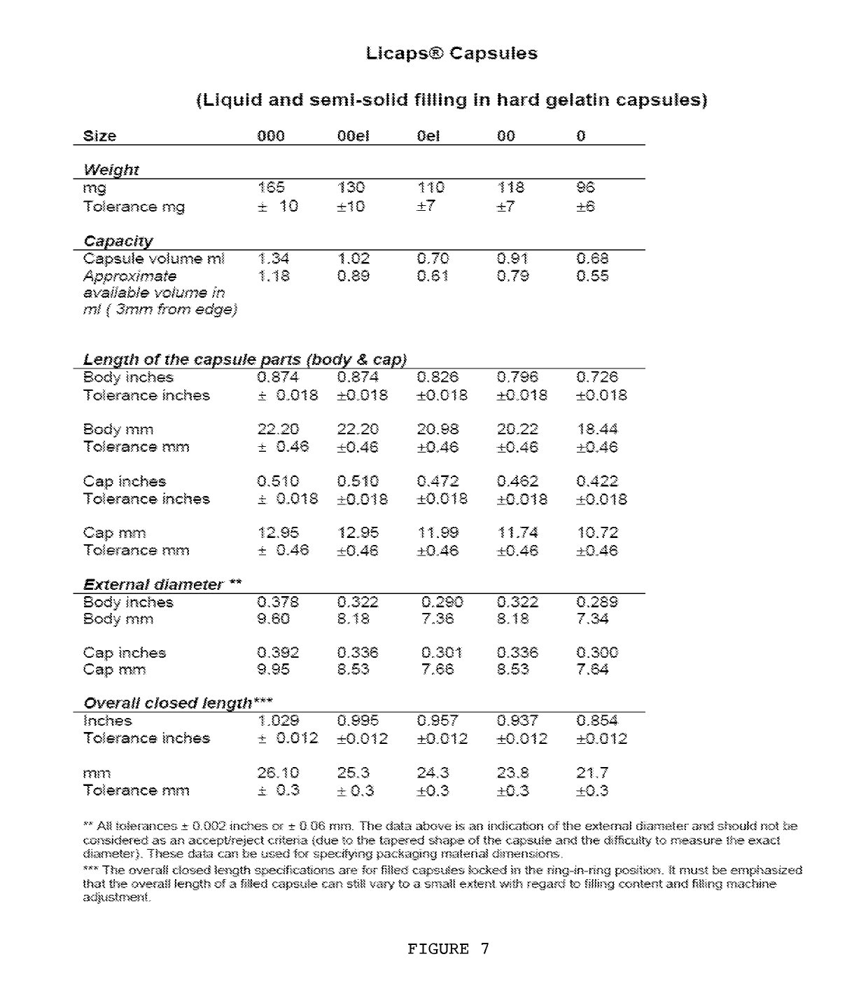

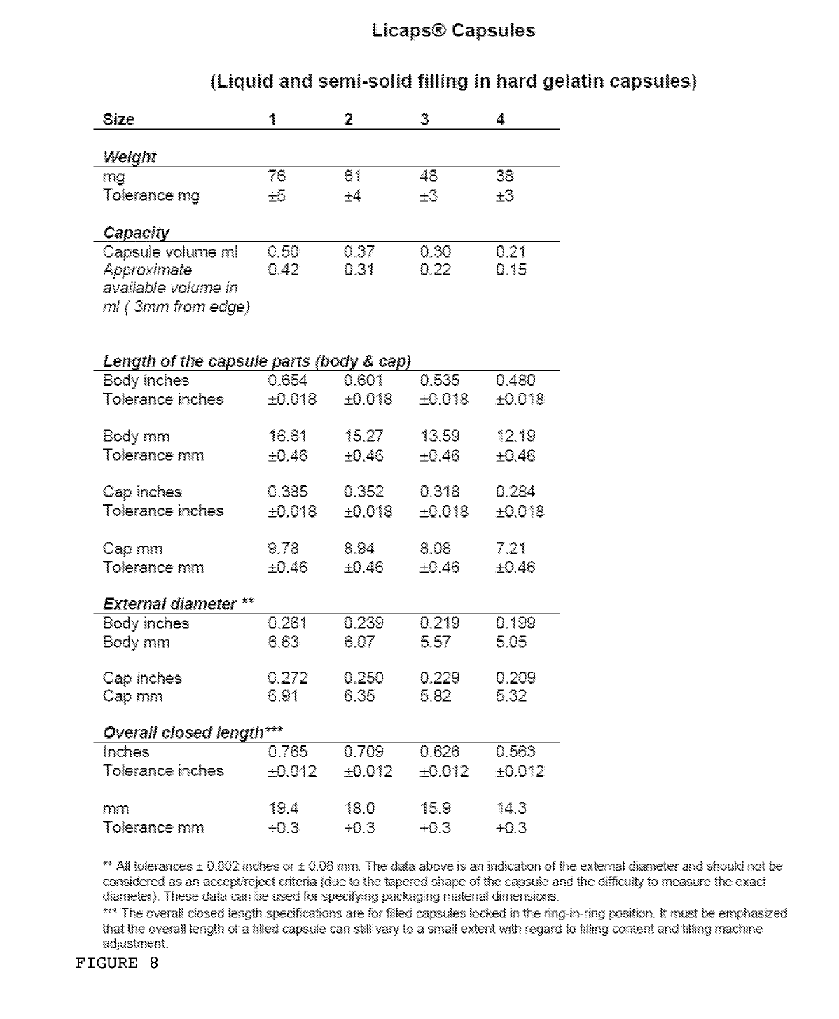

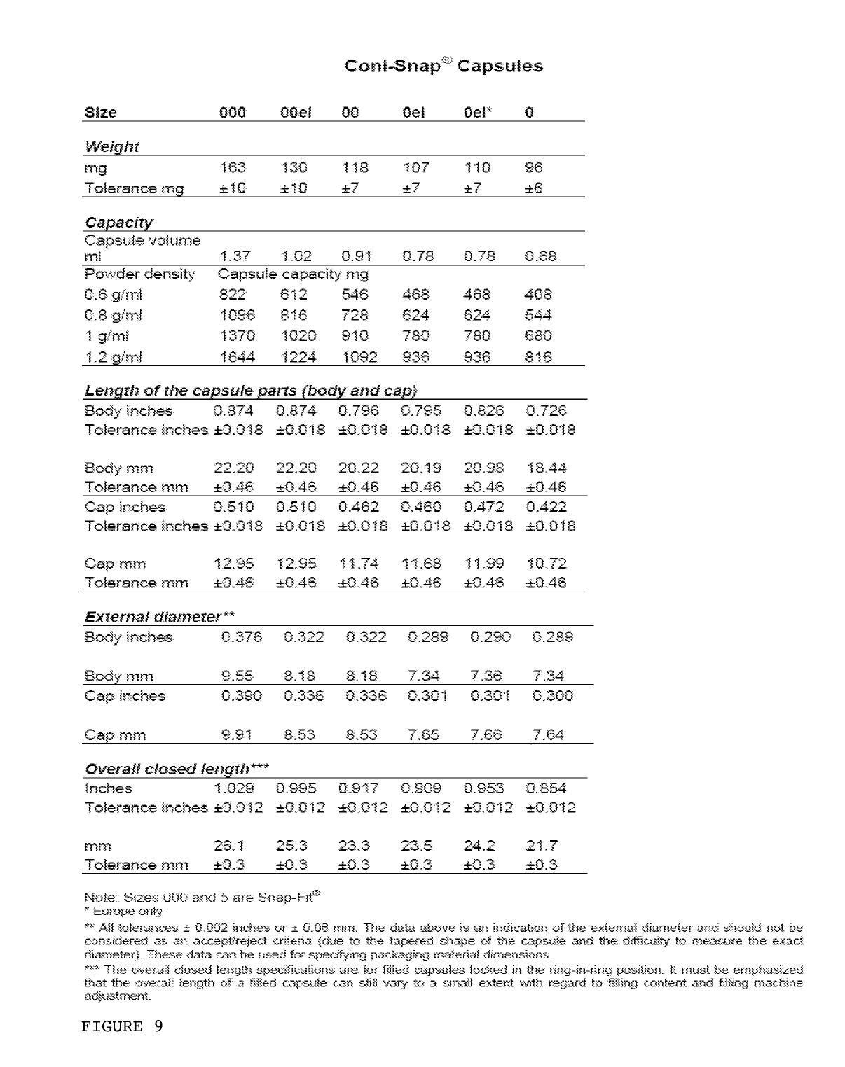

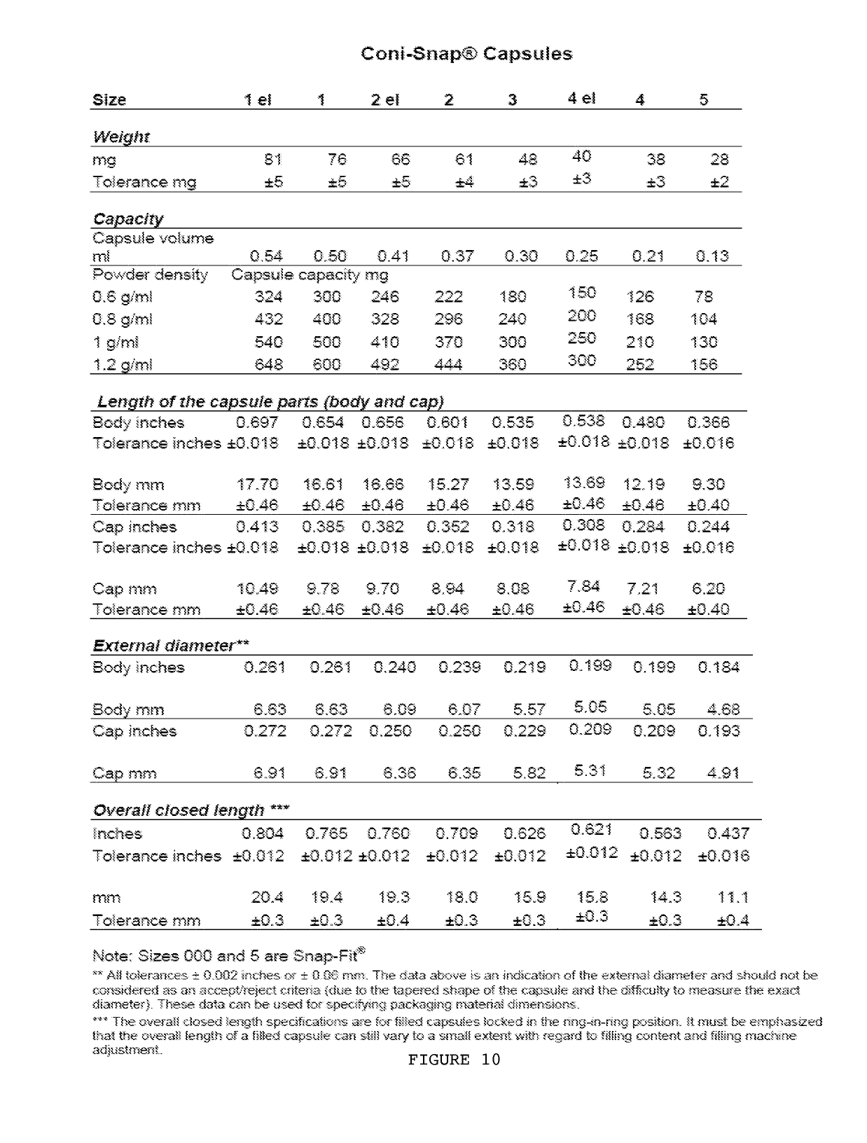

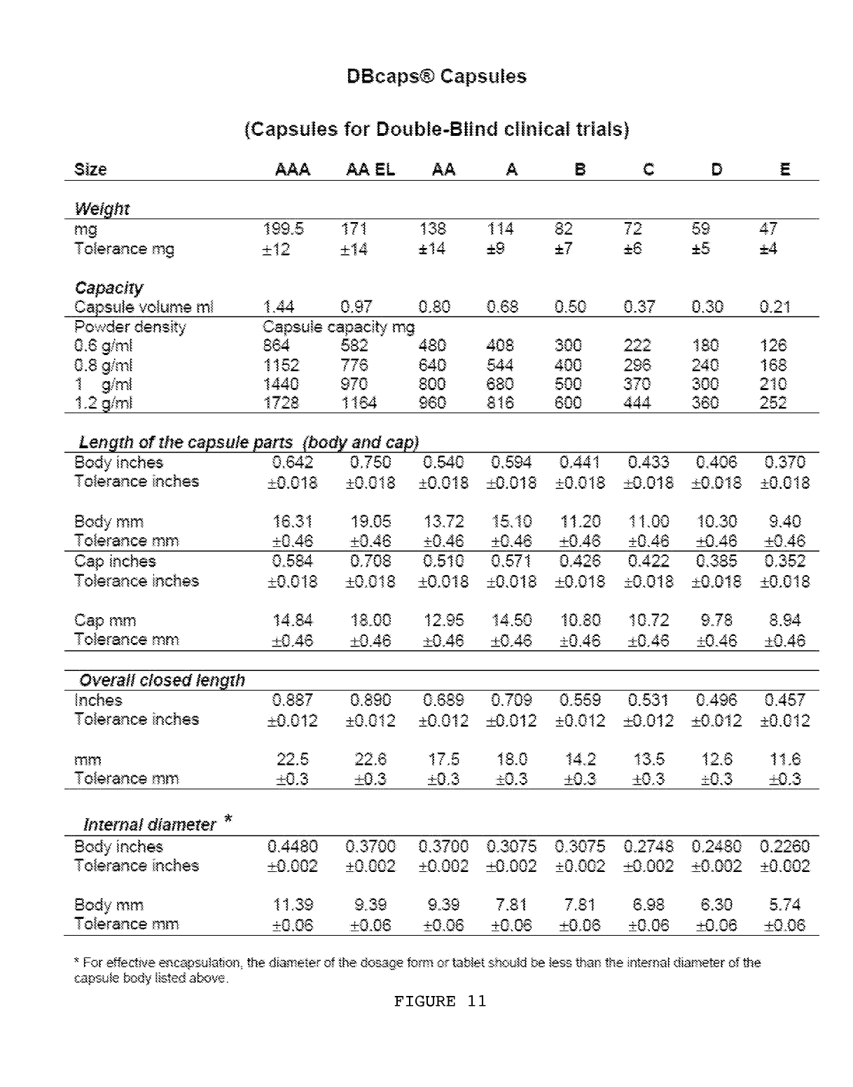

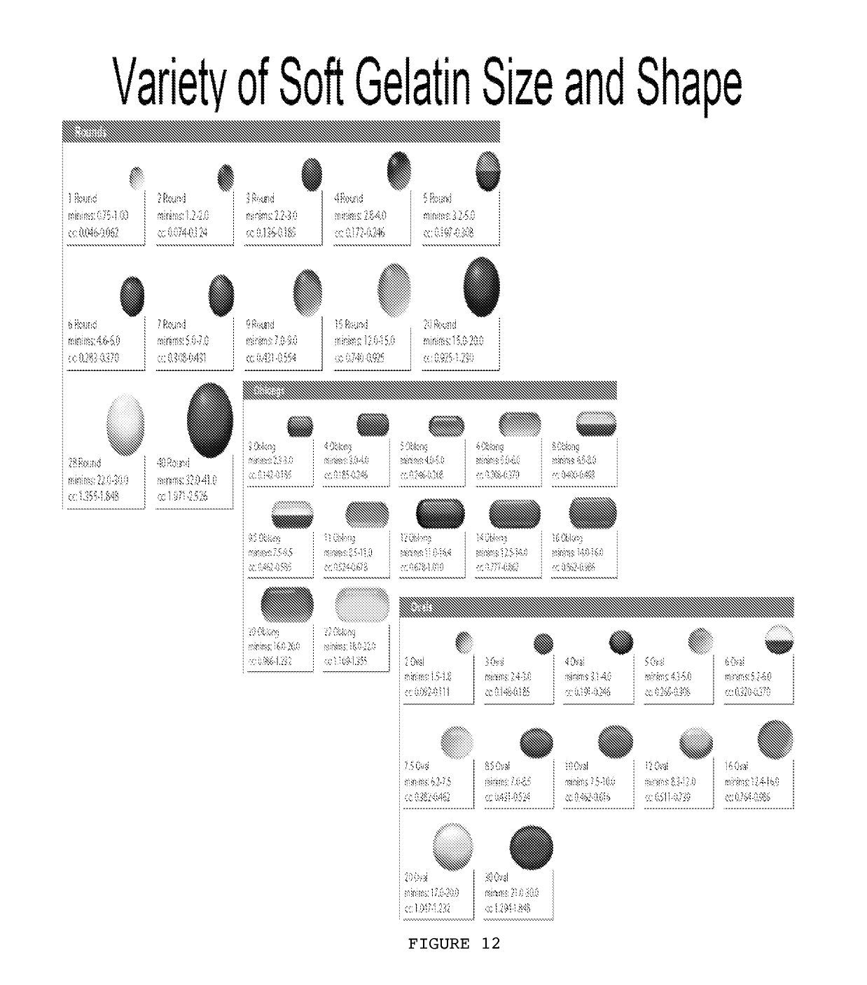

As shown in FIG. 6, a wide variety of shape of SODFs exist. The most common types for CTMs or marketed drugs include round, oblong, and oval. In terms of SMART™ adherence and packaging of CTMs and marketed drugs, the preferred embodiment of SODF shape is oblong, because this shape can be easily accommodated in the various types of capsules, including that they easily fit into the various types of typical capsules (e.g., Capsugel, Peapack, N.J.) such as Licaps® (FIGS. 7 and 8), Coni-snaps® (FIGS. 9 and 10), and DBcaps® (FIG. 11). However, it should be noted that other drug shapes, including but not limited to oval and round, can be readily accommodated with the different types of capsules.

### C.4. Sizing Considerations with SMART™ Formulation Carpentry

FIGS. 2 and 3 show how various types of conventional or standard (tablet-based and capsule-based) SODFs can be packaged with optimized taggant mixtures (e.g., one formulation depicted in FIG. 1), to provide optimal SMART™ adherence function without altering the CMC or the CTM of marketed drugs. Given the sizes listed for the various capsules, including Licaps (FIGS. 7 and 8), Coni-snaps (FIGS. 9 and 10), and DB caps (FIG. 11), and knowing the required volume of the taggant mixture (either in liquid form or bound to particles) along with the dimensions (size and shape) of the CTM or marketed drug (tablet or capsule), it is easy to assemble the pieces into robust new “SMART™” medications that can self report medication adherence. By examining the architecture of Options A to E for tablet-based SODFs and Options F to L for capsule-based SODFs, a number of viable formulation options exist. Ideally, when assembling these medication component elements together, in a preferred embodiment, the final SMART™ mediation contains the least amount of “empty” space possible in the final assembly. For example, if the goal is Option H (FIG. 3) with a specific marketed capsule-based SODF, by knowing the dimensions of this marketed drug, the smallest LiCap (see FIGS. 7 and 8) that could accommodate it is selected and sealed. In turn, typically, the next sized up Licap is selected (see figure) and the 180 μL of taggant mixture (FIG. 1) is placed into this outer Licap, and the smaller Licap is inserted into the larger one, and the larger Licap is sealed.

It will be appreciated that it may be preferable to protect the marketed drug, New Chemical Entity (NCE), Active Pharmaceutical Ingredient (API) or the like from the marker/taggant of the SMART™ system. The LiCap is used for this purpose. In addition, it will be appreciated that by including the taggant, the marketed drug/NCE/API, or each of these separately in particles (whether these are in macro, micro or nanoparticles), an additional degree of separation can thereby be achieved. Where a marketed drug has already received regulatory approval in a given dosage form, it is preferred to leave that dosage form unaltered, and to encapsulate or otherwise separate, by means of LiCap barrier technology described herein, inclusion in particles, or by equivalent means, separate the SMART™ taggant or marker which is the EDIM or which gives rise to the EDIM on metabolism, from the API.

In general, simply going up one size in the LiCap series (e.g., 4 to 3, 2 to 1, etc), easily provides enough volume to hold the liquid taggant (volume of taggant mixture=180 μL) illustrated in FIG. 1. Alternately, two sizes are skipped to accommodate even more volume. This strategy is repeated for all the SMART™ medication assemblies listed in FIGS. 2 and 3. Additional descriptions of these SMART™ medication assemblies are provided in the figure legends. It should be noted that by using Softgel (FIG. 12) to contain the liquid taggant mixture, a tremendous diversity of sizes and shape can be used to package GRAS flavorant taggants with tablet- or capsules-based SODFs (e.g., options A, B, F, G, H).

### C.5. Specific Embodiments of the Invention

The invention covers a wide variety of elements that constitute a SMART™ medication, which self reports its medication adherence. The specific elements are listed below:

C.5.1. Types of Solid Oral Dosage Form (SODF) Containing the API that are Packaged for Definitive Adherence Using SMART™

As already described, the two most important/common SODFs are tablet- and capsule-based systems.

1. Capsule-based type of SODF: API is contained within a capsule manufactured from hard gelatin, softgel, or vegetable (e.g., hydroxypropyl methylcellulose [HPMC]) materials that dissolves in the gastrointestinal tract (GIT), especially the stomach. However, the invention includes capsules, where they are designed, to dissolve in non-stomach areas of the GIT (e.g., enteric coated), including but not limited to the small intestine (e.g., duodenum, jejunum).

a. Vegetarian capsules made of HPMC (hydroxypropyl methylcellulose) such as Vcaps®, Vcaps® Plus, or DRcaps, made by Capsugel (Peapack, N.J.)

b. Hard gelatin capsules made of hard gelatin such as Licaps, Coni-snaps, DBcaps, etc.

2. Hard tablet-based type of SODF

3. Orally Disintegrating Tablet (ODT) type of SODF—Although the focus of this invention is on standard SODFs (tablet or capsule-based), the SMART™ adherence system will work very well in ODTs, and in fact this specific SODF confers unique advantages (see Section D for details).

C.5.2. Release Characteristics of the API from the SODF
Immediate release (IR): this is the most common form of SODF.
Time release: also known as sustained-release (SR), sustained-action (SA), extended-release (ER, XR, or XL), time-release or timed-release, controlled-release (CR), modified release (MR), or continuous-release (CR). In this embodiment, because of technological difficulties in creating time release SODFs, the fact we are using a formulation strategy that does not change or alter the CMC, is highly advantageous. In other words, a major advantage of SMART™ formulation strategy is with time release medications, because of the difficulty of developing suitable time release technologies and the fact any reformulation will alter the release of the API from the drug, alters its PK, and thus its BE, making it unsuitable from a regulatory perspective. Time-release medications can occur in various forms, including capsules, or hard tablets. SMART™ can render time release SODF monitorable from an adherence perspective with no impact on CMC, independent of whether the active ingredient is embedded in a matrix of insoluble substances (e.g., acrylics, chitin), enclosed in polymer-based tablets using laser hole technologies, or microencapsulation technologies.
Enteric Coated (EC): According to USP definition, an enteric coated capsule needs to maintain 100 percent non-disintegration in the first two hours.

### D. SMART™ Adherence and SODF—Orally Disintegrating Tablets (ODTs)

In addition to tablet- and capsule-based SODFs, another very attractive form of SODF for SMART™ adherence is the orally disintegrating tablet (ODT), where absorption is typically rapid and occurs in buccal membranes and/or sublingual areas. Because of a number of unique advantages, ODTs are becoming an increasingly popular drug dosage form. In fact, virtually every major therapeutic class of drugs now contains ODTs. The vast majority of ODTs contain GRAS flavorants (typically high boiling point, low volatility compounds) to mask the bitter taste of the API when they rapidly dissolve in the mouth. Examples of widely used flavorants in medications (e.g., ODTs), are depicted in Table 1 below.

The main advantage of flavored ODTs is that they are already “smart” since they contain an innocuous chemical (GRAS flavorant) that may provide a breath marker that can be used to document adherence. In other words, no additional CMC changes or re-packaging of the CTM or marketed drug would be required for the majority of ODTs in order to document adherence. Furthermore, because ODTs typically dissolve rapidly when placed in the mouth (cannot be easily diverted or “spit out”), the immediate (on order of seconds) appearance of GRAS taggants in breath after dissolution of the ODT indicates adherence in a definitive manner when coupled to simple biometric authentication (e.g., facial recognition) built into the SMART™ device.

TABLE 1
Key Physicochemical Properties of GRAS Flavorants Used in ODTs

| | | | | | | MP |
|---|---|---|---|---|---|---|
| Food Additive | Taste | CAS | Structure | MF | MW | (° C.) |
| vanillin | Vanilla | 121-33-5 | 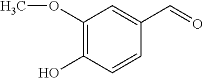 | C₈H₈O₃ | 152.15 | 81.5 |
| benzaldehyde | Cherry | 100-52-7 | 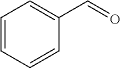 | C₇H₆O | 106.12 | −26 |
| methyl anthranilate | Grape | 134-20-3 | 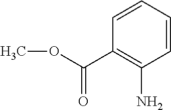 | C₈H₉NO₂ | 151.16 | 24.5 |
| methyl salicylate | Mint | 119-36-8 | 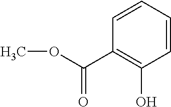 | C₈H₈O₃ | 152.15 | −8 |
| DL-menthol | Peppermint | 1490-84-6 | 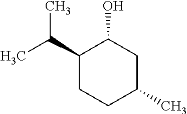 | C₁₀H₂₀O | 156.27 | 35 |
| D-Limonene | Orange (Citrus Oil) | 5989-27-5 | 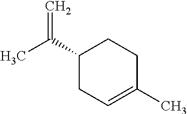 | C₁₀H₁₆ | 136.24 | −74 |
| L-carvone | Spearmint | 6485-40-1 | 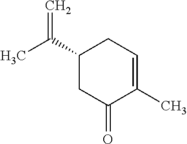 | C₁₀H₁₄O | 150.22 | <25 |
| propofol | IV Anesthetic | 2078-54-8 | 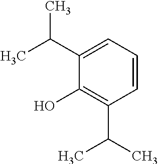 | C₁₂H₁₈O | 178.27 | 18 |
| | | BP | | Water Sol | VP | KH |
| | Food Additive | (° C.) | log P | (mg/L) | (mmHg) | (=CLiq/Cgas) |
| | vanillin | 285 | 1.21 | 11,000 @ 25° C. | 1.18E-04 @ 25° C. | 11,372,093 (EST) |
| | benzaldehyde | 179 | 1.48 | 6,570 @ 25° C. | 1.27 @ 25° C. | 916 |
| | methyl anthranilate | 256 | 1.88 | 2,850 @ 25° C. | 0.0158 @ 25° C. (EST) | 1,987,805 (EST) |
| | methyl salicylate | 223 | 2.55 | 700 @ 30° C. | 0.0343 @ 25° C. | 249 (EST) |
| | DL-menthol | 216 | 3.1 | 456 @ 25° C. | 0.0637 @ 25° C. | 6,736 (EST) |
| | D-Limonene | 176 | 3.537 | 13.8 @ 25° C. | 1.44-1.98 @ 25° C. | 0.94 (EST) |
| | L-carvone | 229 | 2.71 | 1,310 @ 25° C. | 0.103 @ 25° C. | 316 (EST) |
| | propofol | 256 | 3.79 | 124 @ 25° C. | 0.00305 @ 25° C. | 11,533 (EST) |

 Data from the National Library of Medicine, ChemIDplus Advanced
 EST, indicates value estimated from sophisticated chemistry structural program (ChemIDplus Advanced); otherwise values are all experimental
 CAS, chemistry abstract number; MF, molecular formula; MW, molecular weight; MP, melting point; lop P, octanol-water coefficient; VP, vapor pressure; KH,
Henry's Law Constant = concentration of analyte in liquid phase (CLiq)/concentration of analyte in gas phase (Cgas)
 Propofol, the most widely used IV anesthetic in the world, is included as a reference compound, because Xhale has designed a SAW sensor to sensitively measure propofol concentrations in human breath.

Table 1 shows key physicochemical properties of 7 higher boiling point GRAS taggants that are widely used as flavorants in medications including ODTs. The ideal taggants and taggant metabolites for definitive adherence using standard tablet- and capsule-based oral medications are GRAS flavorants with lower boiling points (<130° C.) such as simple aliphatic alcohols (e.g., 2-butanol, 2-pentanol) and ketones (e.g., 2-butanone, 2-pentanone). These are best measured by mGC-MOS. In contrast, for various technical reasons, the mGC-MOS is not suitable for the higher boiling point GRAS flavorants listed in Table 1. For this group of compounds, a SAW-based sensing technology is ideal for SMART™ adherence. Four key elements of this technology yield superior chemical selectivity to distinguish between the various GRAS flavorants: 1) selective sorbent for GRAS flavorants, 2) thermal desorption (i.e., chromatography, 3) selective SAW sensor coatings for GRAS flavorants, and 4) multi-sensor signal processing. SAW sensors to detect other high boiling point compounds such as chemical warfare agents (e.g., nerve gases, blistering agents, mustard agents) are now deployed throughout the world and are highly robust (e.g., accurate, cost effective, portable, durable, low power consumption). For example, a portable SAW sensor to accurately measure breath propofol concentrations (0.1 ppb LOD) to determine minute-to-minute blood levels of this widely used anesthetic (Table 1) is in development with a SAW based sensor similar to the chemical warfare agent detectors marketed by Mine Safety Appliances (HAZMATCAD; http://www.msanorthamerica.com/catalog/product16620.html). Although a SAW-based SMART™ devices for the adherence system can be designed to have a very small logistical “footprint” (e.g., cell phone size), the size of the SMART™ device may be a desktop model sized device (approximately 2″H×4″W×6″L).

With regard to the development of ODT technology, a number of companies have been pioneers and early innovators. Depending upon the physicochemical properties (e.g., Henry's law constant) and the mass of GRAS flavorant incorporated into the ODT, those skilled in the art are able to develop SAW-based SMART™ adherence systems for most types of flavored ODTs. By understanding the role of medication adherence in the variability of the dose-response relationship, it is anticipated that this personalized medicine tool should be useful to a wide variety of companies in several types of drug development programs. Specifically, it is feasible to measure definitive adherence to a wide variety of ODTs, without altering the CMC of the CTM. SAW technology can measure the quantities of most flavorants in ODTs into the gas phase (breath) upon dissolution of the ODT in the sublingual space of the oral cavity. Most ODTs contain between approximately 30-500 μg of flavorants (Table 1). We have already demonstrated in early pilot studies that 30 and 500 μg of L-carvone, methyl salicylate, methyl anthranilate, and benzaldehyde dissolved in 50 μL of neat ethanol were easily detected in human breath by a SAW sensor with polymer coatings optimized for propofol (not optimized for these GRAS flavorants) after being placed on the surface of the tongue. In contrast, the same mass of vanillin gave a weak SAW response. This finding should not be surprising, given the SAW polymers were not optimized for these analytes and the adverse physiochemical properties of vanillin. Specifically, in terms of being measured in the breath (gas phase), of the 7 GRAS flavorants used in ODTs listed in Table 1, vanillin has by far the most adverse Henry's Law constant (K_{H}=11,372,093). Thus, vanillin, given its physiochemical characteristics (e.g., high water solubility, low log P, very low vapor pressure), has an overwhelming predisposition to remain in the liquid phase, when placed in the mouth, and not escape to the gas phase (see below for further discussion). Last, it should be noted that the propofol SAW sensors easily detected the ingestion of Tylenol cool caplets, spearmint tic tacs, freshmint tic tacs, cinnamon tic tacs, and orange tic tacs, which contain DL-menthol, L-carvone, DL-menthol, cinnamaldehyde, and D-limonene, respectively, after being placed in the mouth.

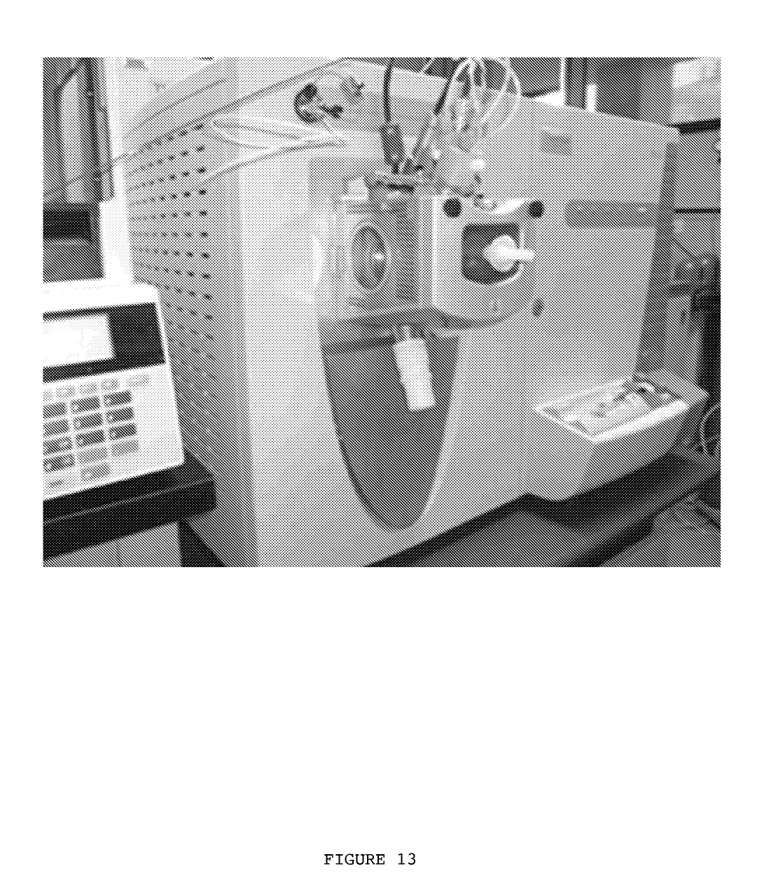

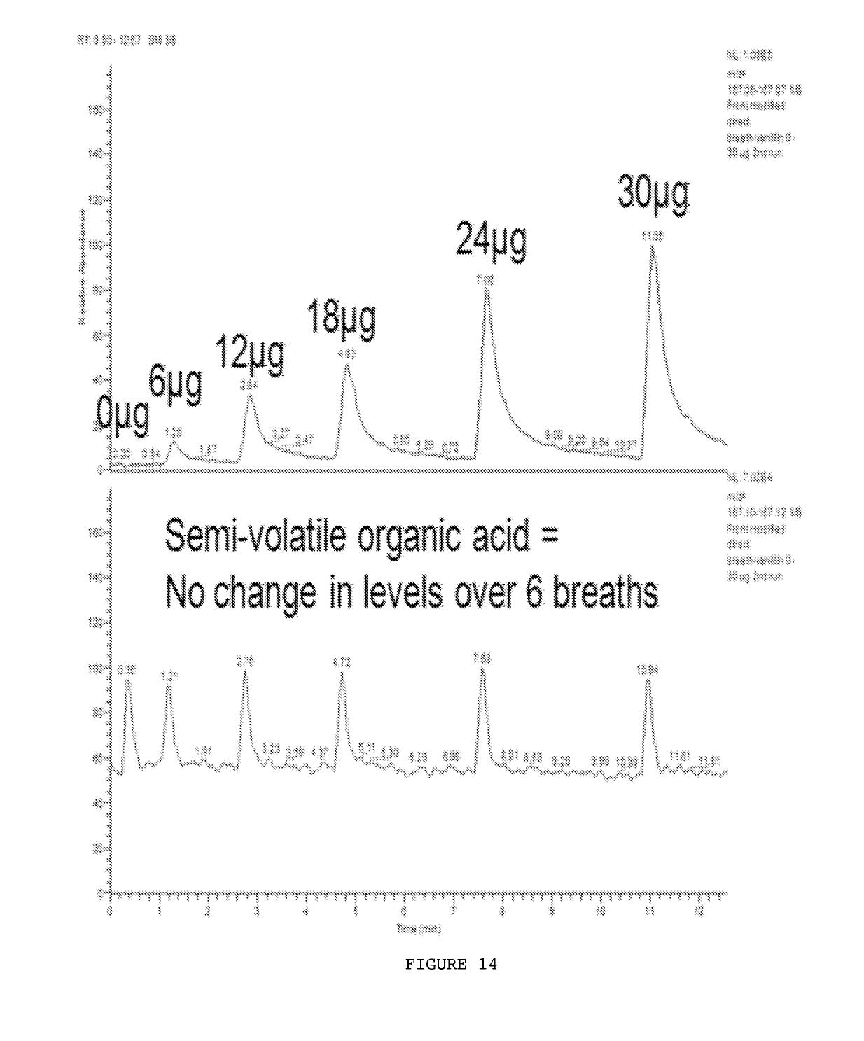

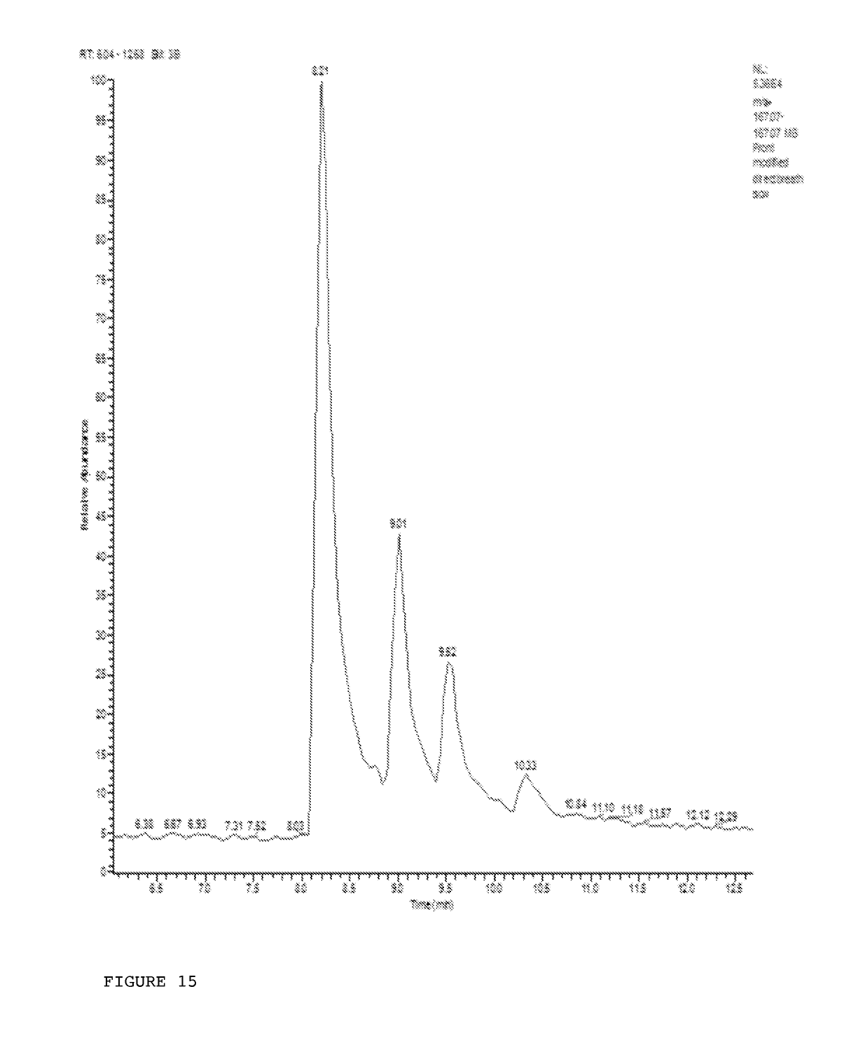

To definitively establish whether relevant doses of vanillin in ODTs, when placed on the tongue, can generate detectable gas phase concentrations in human breath, a $1 M mass spectrometer (OrbiTrap, FIG. 13) was utilized, which allows direct exhalation into the device for breath analysis. In the first set of experiments, we established that vanillin generates a dose (0, 6, 12, 18, 24, and 30 μg dissolved in 10 μL neat ethanol)-dependent increase in gas phase concentrations of the flavorant in human breath (upper panel, FIG. 14). In the lower panel of FIG. 14, note how an internal standard (semi-volatile organic acid) stays constant for each of the six breaths. In a second set of experiments, we investigated how long vanillin (30 μg dissolved in 10 μL neat ethanol) persists in human breath after being placed on the tongue (FIG. 15). Vanillin appears to persist in human breath for ≈2 min.

Taken together, these findings indicate that it is feasible to develop a viable SAW-based medication adherence system, even at lower doses of the GRAS flavorants listed in Table 1 when packaged into ODTs.

In light of the foregoing general description, it will be appreciated that this invention comprises at least the following specific embodiments:

A Solid Oral Dosage Form (SODF) comprising a marker composition and an Active Pharmaceutical Ingredient (API) wherein the marker composition and the API are not in direct contact with each other. Preferably, the marker composition comprises a directly detectable Exhaled Drug Ingestion Marker (EDIM), or a marker which is metabolically converted into an EDIM, or both. In a preferred embodiment according to this invention, the SODF comprises both the directly detectable EDIM and a marker which is converted into an EDIM following metabolic activity following ingestion of the SODF.

Preferably, the SODF comprises either (a) a tablet comprising the API, (b) a capsule comprising the API, or (c) particles containing the API, while the marker composition is present in a format selected from the group consisting of: (a) a tablet (b) a coating surrounding the API (c) a capsule (d) loose particles (e) particles contained within a tablet (f) particles contained within a capsule (g) particles surrounding the API wherein the marker particles and the API are contained within a capsule which contains both and (h) combinations thereof.

In particularly preferred embodiments according to this invention, the SODF has a form selected from any of the forms shown in FIG. 2 or 3.

In a preferred embodiment according to this invention, the marker comprises either a flavorant which gives rise to an Exhaled Drug Ingestion Marker (EDIM) if the SODF is an Orally Disintegrating Tablet, (ODT) or, if not an ODT, the marker comprises at least one secondary alcohol and at least one ketone for definitive medication adherence monitoring, wherein the secondary alcohol and the ketone are each non-toxic at the dosage included in the SODF. In such an embodiment, the ketone is directly detectable in exhaled breath of a subject as an Exhaled Drug Ingestion Marker (EDIM) and the secondary alcohol is detectable as an EDIM following metabolism to a ketone metabolite of the alcohol. Preferably, the secondary alcohol is selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-mthyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol. Preferably, the ketone is the ketone of a secondary alcohol selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-mthyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol.

In an embodiment where the SODF is an ODT, the marker is selected from the group consisting of vanillin, ethyl vanillin, cinnamaldehyde, benzaldehyde, methyl anthranilate, methyl salicylate, menthone, DL-menthol, D-limonene, L-carvone, or combinations thereof. It will be appreciate that unless excluded herein, material included in a first embodiment may be included in any other embodiment.

Additionally, it will be appreciated that several hard tablets and/or capsules can be packaged into a single SMART™ drug (e.g. a “Life Style” medication containing, e.g. a statin, a blood pressure medication and aspirin). This is particularly advantageous is some subpopulations were cognitive function is impaired and adherence to multiple medications must be monitored on a daily (or regular) basis.

It will likewise be appreciated that a method according to this invention utilizes the SODF as described above for monitoring subject adherence with a medication regimen. This involves:

(i) providing to the subject a Solid Oral Dosage Form (SODF) comprising a marker composition and an Active Pharmaceutical Ingredient (API) wherein the marker composition and the API are not in direct contact with each other. The marker is either directly detectable in exhaled breath of a subject as an Exhaled Drug Ingestion Marker (EDIM) or it is metabolically converted into an EDIM, or both; and

(ii) monitoring the exhaled breath of said subject to detect said directly detectable EDIM or said metabolically produced EDIM or both.

In practicing such a method, those skilled in the art will appreciate that various preferred embodiments of the SODF as described above may be employed.

The method according to this invention may be practiced with a SODF comprising several hard tablets and/or capsules packaged into a single SMART™ drug (e.g. a “Life Style” medication containing, e.g. a statin, a blood pressure medication and aspirin). In such an embodiment according to the invention, the ratio of a directly detectable EDIM to an EDIM produced upon metabolic activity may be monitored to obtain unique additional information about adherence and metabolic state of the subject.

To ensure that the specifics provided herein below in the Examples section, as well as with respect to specific embodiments mentioned herein above, are not incorrectly interpreted as limiting on the invention, the following additional considerations are mentioned here:

Additives to various dosage forms, in addition to the adherence enabling markers (AEMs), may be needed as excipients to stabilize the AEM formulation in the SMART™ SODFs by providing a thickener/binder function. Examples of typical thickeners/binders that are standard and widely used in pharmaceutical formulations include but are not limited to those listed in the FDA inactive ingredients (IIG) database published by the U.S. Food and Drug Administration (http://www.fda.gov/Drugs/InformationOnDrugs/ucm113978.htm) or the United States Pharmacopeia (USP; http://www.usp.org/usp-nf). Preferred examples of excipients that could serve as binders/thickeners include but are not limited to the cellulose ether polymers, including carboxymethyl cellulose (CMC), carboxymethyl hydroxyethyl cellulose (CMHEC), hydroxyethyl carboxymethyl cellulose (HECMC), hydroxyethyl cellulose (HEC), hydroxypropyl cellulose (HPC), hydroxypropyl methylcelluose (HPMC), methylcellulose (MC), methyl hydroxyethyl cellulose (MHEC), and methyl hydroxypropyl cellulose (MHPC).

In general, therefore, those skilled in the art, based on the present disclosure, will appreciate that SODFs according to this invention may include any excipients that ensure that marker composition and the API do not come into direct contact with each other while in the SODF, provided, also, that they enhance stability and/or compatibility within the final dosage form, and preserve and/or enhance adherence monitoring function of markers. The AEMs included in the SODFs according to this invention are preferably generally recognized as safe compounds, (GRAS compounds), including but not limited to alcohols and ketones, preferably selected from secondary alcohols, tertiary alcohols, and secondary ketones. In addition, those skilled in the art will appreciate that these markers may include non-ordinary (but preferably non-radioactive) isotopes, including, but not limited to deuterated markers, and markers containing non-ordinary isotopes of oxygen, carbon, nitrogen and the like. Of course, for certain specific applications, even radioactive markers could be used, but naturally, for general consumption during clinical trials, and in any other format of generalized adherence monitoring, (including but not limited to adherence monitoring for pharmaceutical products, including use in clinical trials and in disease management and prevention, drug diversion, drug interactions and assessments of drug metabolism, anti-counterfeiting, gastric emptying, therapeutic drug monitoring, including for oral delivery of AEMs or Adherence Formulations via capsules, including liquid or solid (e.g., powder) AEMs or Adherence Formulations, AEMs or Adherence Formulations embedded in capsule shells, and/or AEMs or Adherence Formulations coated on, sprayed, and or/applied to the exterior of capsules.

It will further be appreciated that use of GRAS compounds as markers does not exclude use of flavorants in ODTs, including SODFs, provided the analytical techniques used to measure the AEMs are not confounded by the inclusion of such flavorants, including for sublingual tablets and chewable tablets in addition to ODTs.

The architectural approaches to SODFs provided herein allow for easy incorporation of marker composition(s) into the SODFs to minimize impact on API and not alter CMC. These techniques and geometries include, but are not limited to, anchoring smaller capsules containing marker compositions to an unfilled portion of a capsule shell. This also allows maximum available volume for filling. Additional architectural approaches to SODFs include but are not limited to a capsule-in-capsule strategy, embedding markers in capsule shells, adding coating containing markers to a tablet or incorporating markers into an existing coating, and coating/spraying on/applying markers to exterior of tablet or capsule.

As shown in the below Examples section, the present disclosure demonstrates the viability of detecting GRAS flavorants (e.g., D-limonene, methyl salicylate, and D-limonene±methyl salicylate) in simulated sublingual (SL) tablets as a means of documenting medication adherence using the SAW sensor.

It will further be appreciated from the present disclosure that secondary (2°) alcohols and 2° ketones, tertiary (3°) alcohols, and combinations thereof may be used as medication adherence markers (AEMs, Adherence Enabling Markers). The preferred 2° alcohols are those that are GRAS compounds, including but not limited to 2-propanol, 2-butanol, 2-pentanol, 3-pentanol, 2-methyl-2-butanol, 3-methyl-2-butanol, 3,3-dimethyl-2-butanol, 3-hexanol, 2-hexanol, 3-methyl-2-pentanol, 4-methyl-2-pentanol, 2,4-Dimethyl-3-pentanol, 2-methyl-3-hexanol, 2,6-dimethyl-4-heptanol, 2-heptanol, 3-heptanol, 4-heptanol, 5-methyl-3-heptanol, 6-methyl-3-heptanol, 2,3,4-trimethyl-3-pentanol, cyclobutanol, cyclopentanol, cyclohexanol, cycloheptanol.

The preferred ketones are those that are GRAS compounds, including but not limited to, e.g. 2-pentanone, and others shown herein in FIGS. 4 and 5.

The preferred 3° alcohols are those that are GRAS compounds including but not limited to tert-butanol (2-methyl-2-propanol), 3-methyl-3-pentanol, 2-methyl-2-pentanol, 2,6-dimethyl-2-heptanol (lolitol).

2-pentanone, for example, when included in the formulation, is exhaled unchanged in the breath (i.e. it does not require metabolism to appear in breath) and serves as a primary marker or as a confirmatory or additional marker in combination with another marker. For example, 2-pentanone serves such a function when used with t-butanol, from which 2-butanone is generated via ADH. 3° alcohols are useful according to this invention in a similar or identical functional capacity as the ketone(s), as the 3° alcohols appear in breath as adherence marker much like the ketones do. Unlike primary and secondary alcohols, tertiary alcohols are not oxidized by Phase 1 processes, and therefore they appear in the breath unchanged. A fraction of the 3° alcohols may be subjected to Phase 2 metabolism (e.g., direct conjugation of the hydroxyl group via glucuronidation), but the majority of the 3° alcohol mass included in adherence compositions is unchanged and appears in the breath. Any tertiary alcohol listed in the food database (Leffingwell & Associates, Flavor-Base Professional, 2007) is useful for this purpose.

Secondary alcohols (see, e.g. FIGS. 4 and 5) included in the authoritative Leffingwell & Associates, Flavor-Base Professional, 2007, may be used, but it will be appreciated that 2° alcohols that are GRAS compounds, are preferred.

GRAS flavorants (including but not limited to those shown in Table 1) are useful for inclusion in ODTs, SL, and chewable tablets. In these scenarios, it will be appreciated that the EDIM is the GRAS flavorant itself. Any compound that is used to provide unique flavors in foods or medicines could be used for this purpose, but those explicitly listed as GRAS (safe for food) are, again, preferred.

Those skilled in the art will appreciate, based on the present disclosure, that there are many slight modifications of the molecular entities listed in Table 1, which could be used to advantage according to the present invention. For example, menthone and menthol, which are in the food databases, are useful for this purpose. The Leffingwell database may be referenced for this purpose. GRAS flavorants that have a boiling point that is equal to or greater than 150° C. are useful for ODTs, SL, and chewable tablets.

When placing the AEM formulation into adherence capsules, referred to herein as “AdhCaps”, it is preferable to place them in areas of the capsules (e.g., Capsugel DB Caps®, LiCaps®, or ConiSnaps®), where the space is wasted anyway. In this way, the pharmaceutical company gets the full volume of the capsule to use for their clinical trial material, and the automated manufacturing filling processes, which have already been developed, still work with the AdhCaps. For example, commonly used capsules used for overencapsulation and blinding clinical trial materials (CTMs) are the size AA DBCap®. Another capsule, gelcap or the like containing the Adherence Enabling Marker, AEM, formulation, whatever its architecture, whether it be a hard or soft gel, is preferably located in the most apical portion of the cap of a size AA DBcap® capsule. Thus, a gelcap containing the AEM dropped into the apical half of a size AA DBcap® capsule, permits the larger, lower portion of the size AA DB capsule to be filled with placebo, API formulation, and the like, without the gelcap negatively impinging on the available fill volume to accommodate the placebo or AlP formulation.

Those skilled in the art will appreciate that where the marker is known to be compatible with the API, then these components may be permitted to contact each other in a given dosage form according to this invention.

While reference is made herein to capsules and the like available from a particular manufacturer, including under trademark names of particular manufacturers, and including specific dimensions, in some cases, those skilled in the art will appreciate that the considerations and disclosure provided herein in that regard is not limiting. Thus, capsules from any manufacturer may be appropriate for use according to this invention, provided that the active therapeutic agent, if present, and the adherence enabling markers, do not interact within the given shelf-life of a given formulation or SODF, and provided that the quality and dimensions of such materials are sufficient to meet the geometric requirements outlined herein and those of appropriate regulatory bodies.

The present disclosure, including but not limited to the exemplary disclosure provided herein, supports at least the following conclusions:

1. GRAS flavorants commonly found in pharmaceutical products can be easily used to document medication adherence using the SMART™ Adherence System.

2. Powders which stably contain many types of GRAS flavorants (e.g., FONA wintergreen powder containing methyl salicylate), are commonly used in the manufacture of many types of pharmaceutical products. These types of powders are frequently employed in the manufacture of flavored pharmaceutical dosage forms, where the GRAS flavorants are stably incorporated into the drug product (e.g., Alayert® Fresh Mint).

3. In particular, GRAS flavorants are frequently employed to impart a flavor that can mask the potentially bitter taste of the active pharmaceutical ingredient (API) in tablets, including but not limited to orally disintegrating tablets (ODTs), sublingual (SL) tablets, and chewable tablets, which are primarily absorbed into the body by dissolving in the mouth. When these tablets (e.g., ODT, SL tablets, chewable tablets) dissolve in the mouth, they rapidly liberate gas phase concentrations of the relevant GRAS flavorant into the breath that are readily detectable by both mass spectroscopy and by a portable sensor device, including but not limited to a portable surface acoustic wave (SAW) sensor device, used in the SMART™ Adherence System.

4. The easy detection of the marker in breath by the portable sensor (e.g., SAW) device in the SMART™ Adherence System indicates that quantities of GRAS flavorants ordinarily placed into tablets (e.g., ODTs) by pharmaceutical companies is sufficient for the system to work. For example, an example illustrates the detection of placement of the Alayert® Fresh Mint ODT in the mouth using the portable SMART™ SAW sensor. In addition, the SAW device could easily detect placement of low mass quantities (suitable for incorporation into a finished pharmaceutical tablet) of the FONA Wintergreen powder after being placed on the surface of the tongue (e.g., detection of methyl salicylate by the SAW sensor).

In summary, these data provide information that the SMART™ Adherence System, which documents medication adherence by detecting markers in breath, is technologically feasible.

### EXAMPLES

Having generally described this invention herein above, including with respect to the best mode of carrying out this invention, the following exemplary supported is provided to further enable those skilled in the art to practice this invention to its full scope. This detailed written description and enabling disclosure is not, however, intended to be limiting on the invention. Rather, for an apprehension of the scope of the present invention, those skilled in the art are directed to the appended claims and their equivalents.

### Example 1

### Use of SAW Detection of D-Limonene and Methyl Salicylate to Document Adherence to Sublingual Medications

### Summary

Self-Monitoring And Reporting Therapeutics (SMART™) is a concept whereby FDA-designated direct food additives (e.g., GRAS flavorants) are coadministered with active pharmaceutical ingredients (APIs) to produce breath markers that can be detected by a handheld sensor to document medication adherence. The breath marker can be either the additive itself or a metabolite of the additive. For orally disintegrating tablets (ODTs) including sublingual (SL) tablets, flavoring agents may function as the entities which produce a breath marker. The objectives of the current study were to assess the feasibility of using a surface acoustic wave (SAW) sensor to document adherence to specifically flavored SL medications, and, if feasible, evaluate its use in a pilot clinical study.

The study contained two specific aims. In Aim 1, the interaction between the SAW sensor and two model flavorants, D-limonene and methyl salicylate, was characterized, and the dose-response relationships and breath kinetics of these flavorants following SL administration of test solutions and powder formulations were determined. Using these results as a guide, three different SMART™ placebo SL formulations (D-limonene, methyl salicylate, D-limonene+methyl salicylate) were designed and prepared. In Aim 2, these three SMART™ placebo SL formulations plus two additional placebo SL formulations containing no volatile flavorants were administered during each study visit to a cohort of eight study participants with each done in triplicate on different days for a total of 24 subject visits and 120 observations in a prospective, double-blind, randomized study. Upon completion of the study, all SAW data were compiled and provided to a blinded researcher who used this information to predict which formulation was given sublingually for each SAW data result.

An optimum SAW sensor configuration (e.g., detector coatings, concentrator packing material) for the simultaneous determination of D-limonene and methyl salicylate was used in this study and was found to maintain excellent sensitivity over time and required only 3 ng of methyl salicylate and 10 ng of D-limonene to detect each flavorant. Compared to D-limonene, the SAW sensors were 3-5 times more sensitive to methyl salicylate, and the methyl salicylate demonstrated greater inherent stability in the SL powder formulations. The high volatility of D-limonene made it necessary to add it to the SL powder in a higher concentration than was initially predicted by solution-based dosing experiments and to incorporate it into the powder immediately before use to prevent loss. Combinations of 30 μg methyl salicylate and 200 μg of D-limonene in each 30 mg aliquot of placebo SL powder were chosen for the final SMART™ SL formulations used in the clinical pilot study (Aim 2).

After reviewing the SAW data from the eight subject clinical study, a blinded researcher was able to correctly identify which SL formulation was given to each subject across all observations (120/120). Despite the large variability in observed breath concentrations of D-limonene and methyl salicylate following SL powder administration, there were no discernible differences in the performance of the SAW sensors and ability to qualitatively identify the different formulations. The handheld SAW sensor demonstrated 100% sensitivity and 100% specificity. The SAW sensor proved to be stable and durable with moderate to heavy sample loads and successfully demonstrated that a flavorant-based SMART™ Adherence System for monitoring adherence to SL medications is feasible.

Note that the tables included in this Example are not continuous in numbering with the remainder of the body of the text, and references to tables herein are to the tables included in this Example rather than those in the remainder of the text of this disclosure.

Details of this Study

The Self-Monitoring and Reporting Therapeutics (SMART™) Adherence System was developed to provide a means whereby exhaled breath is used to monitor medication adherence. In the SMART™ Adherence System, specific FDA-designated direct food additives (e.g., GRAS flavorants) are coadministered with an active pharmaceutical ingredient (API). Once introduced in the oral cavity or absorbed by the body, these flavorants generate markers that appear in breath, either as the flavorant itself or a metabolite of the flavorant, and thereby serve as markers of adherence to the API. To date, prototype SMART™ versions of drugs using capsules, tablets, orally disintegrating tablets (ODTs), and topical gels have been created and investigated.

Sublingual (SL) tablets are a class of ODTs that deliver drugs systemically via the mucosa lining the floor of the mouth. SL tablets are often flavored to mask the bitter taste of the API and improve acceptability to the patient. Upon dissolution of the SL tablet, these flavorants are released (along with the API) and will persist in the mouth for variable times, depending on the physicochemical characteristics of the flavorant. Specifically, the flavorant gas phase concentration in the oral cavity will depend on its Henry's Law constant (i.e., K_{H}; gas to liquid phase partitioning due to key physicochemical factors, including volatility, ionization, lipophilicity, and water solubility). Since these flavorants are released with the API, it was recognized that they could potentially function as SMART™ adherence markers.

The Xhale surface acoustic wave (SAW) sensor was developed to measure the concentration of semi-volatile compounds in gaseous samples ranging from ambient air to exhaled human breath. In order to impart both taste and smell sensations, flavoring agents are often semi-volatile compounds. The SAW sensor can detect trace (part per billion) levels of several commonly used flavorants (e.g. methyl salicylate and methyl anthranilate). Given this level of sensitivity, the incorporation of microgram amounts of flavorants with proper K_{H} values into the SL matrix was expected to generate a reliably detectable SAW signal.

D-limonene and methyl salicylate are considered to be interesting marker compounds because their physicochemical properties allow for their detection by the SAW sensor in the breath. It was therefore considered by the present inventors to be theoretically possible to create characteristic breath patterns using appropriate doses of these two flavorants that would unambiguously indicate use of a particular SL tablet.

The purpose of the current study was to conduct an initial investigation of the feasibility of D-limonene and methyl salicylate as potential adherence markers for SL tablets and to establish the feasibility of using a SAW-based system for monitoring adherence to SL medications in a clinical study.

### Materials and Methods

### Test Articles and Formulations

Methanol (HPLC grade) was purchased from Fisher Scientific (Lot #096609). Ethanol (USP grade) was purchased from Fisher Scientific (manufactured by AAPER Alcohol and Chemical Co., Shelbyville, Ky., Lot #07A3023). Vanillin (4-hydroxy-3-methoxybenzaldehyde) was purchased from SAFC, St. Louis, Mo. (Lot #MKBG1356V). Methyl Salicylate (methyl-2-hydroxybenzoate, CAS 119-36-8) was purchased from SAFC, St. Louis, Mo. (Lot #MKBG1335V). D-limonene (4-isopropenyl-1-methylcyclohexene, CAS 5989-27-5) was purchased from SAFC, St. Louis, Mo. (Lot #MKBB4944V). Placebo SL powder matrix included standard, widely-used excipients in these types of fomulations. The SL matrix was compounded with and without vanillin by a certified pharmacy, Westlab Pharmacy (Gainesville, Fla.).

### Study Sites

All studies were performed in the Nanoscale Research Facility at the University of Florida, Gainesville, Fla. After approval by the Western Institutional Review Board (WIRB), human study participants were consented and enrolled in Aim 1 (protocol 20100140) and Aim 2 (protocol 20120658).

### Instrumentation

Four prototype SAW devices were utilized in these studies. As illustrated in Table 1, each SAW device was configured with identical concentrators, packing materials and proprietary detector surface coatings. This configuration of concentrator, packing and surface coatings was selected for its ability to detect nanogram quantities of both flavorants and separate D-limonene from methyl salicylate. Each unit was identical in terms of sampling flow rates, temperature program, and cycle times. SAW device 1 was used for all studies in Aim 1 and a portion of those in Aim 2. Devices 2, 3, and 4 were used only in Aim 2.

TABLE 1
Configuration and operational parameters of the SAW sensor

| Parameter | Value |
|---|---|
| Concentrator Packing | Carbopack Y |
| Detector 1 Surface | Polymer “EI” (hydrogen-bond/polar |
| Coating | interactions) |
| Detector 2 Surface | Polymer “OL” (aromatic/dipole- |
| Coating | dipole interactions) |
| Sampling flow rate | 500 std. cu. cm/min |
| Cycle Time | 45 seconds |

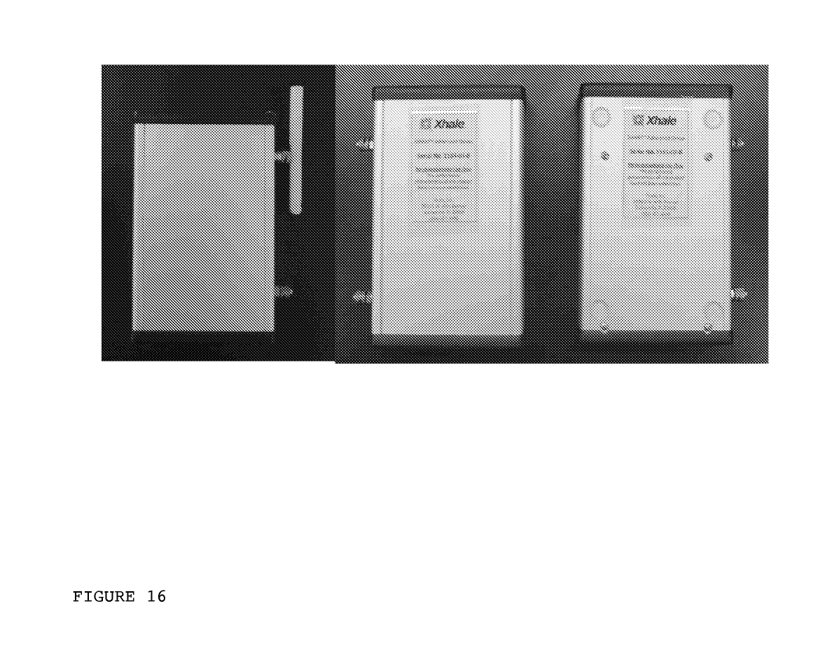

Liquid chromatography mass spectrometry was performed using a Perkin Elmer (Waltham, Mass.) series 200 LC system coupled to a Thermo Scientific (Waltham, Mass.) LTQ Orbitrap XL, (FIG. 16)

### Preparation of Standard and Spiking Solutions

Stock solutions of flavorants were prepared by weighing out desired amounts of neat D-limonene or methyl salicylate into 50 mL volumetric flasks on a calibrated analytical balance and diluting to volume with USP ethanol. These stock solutions were transferred to 40 mL vials for storage. Standard and spiking solutions were prepared from these stock solutions by serial dilution using calibrated pipettes. Stock, standard, and spiking solutions were stored in air-tight vials at 4° C. after preparation.

### Calibration of the SAW Sensor and Measurement of Detector Response

SAW sensors were calibrated by injecting known masses of D-limonene and methyl salicylate directly into the concentrator inlet. One μL (1 μL) aliquots of D-limonene and methyl salicylate standards ranging in concentration from 3-300 μg/mL (dissolved in methanol) were injected to generate calibration curves corresponding to 3-300 ng of each flavorant being introduced to the trap.

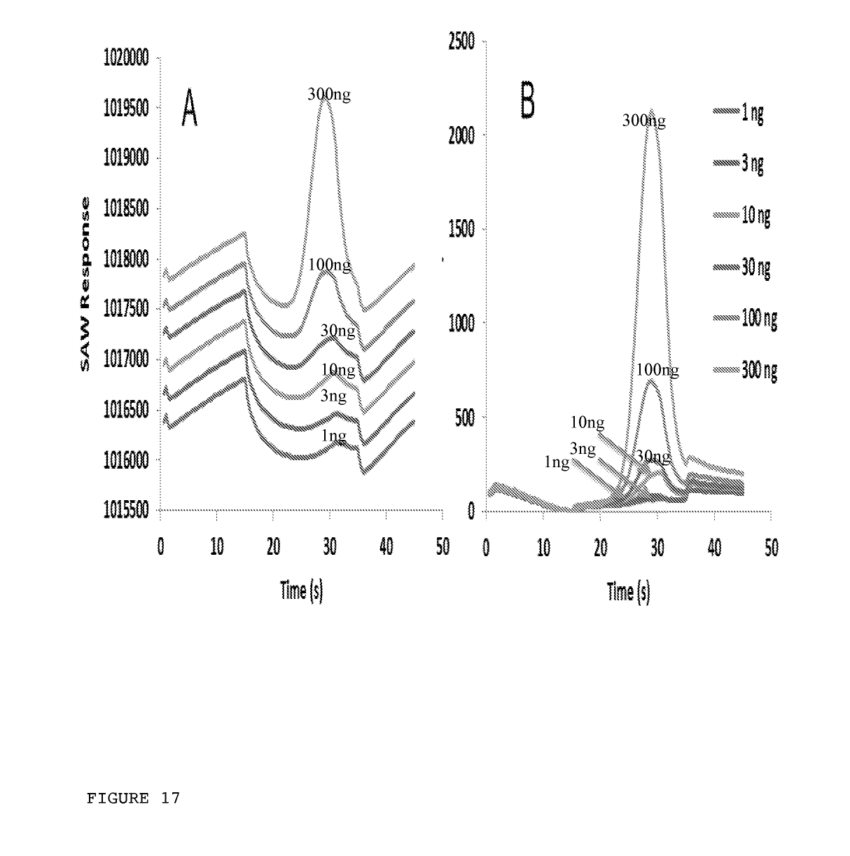

In addition to measuring flavorants and other volatile compounds, the two SAW detectors housed within the sensor respond to changes in flow rate produced by the opening and closing of valves during the sampling run. These variations produce reproducible artifacts in the raw detector output for all samples. As illustrated in FIG. 17, to isolate the response produced by compounds of interest from these background artifacts, blank air runs were routinely subtracted from standard runs.

The height of peaks remaining in the baseline-subtracted SAW traces were measured to determine standard responses.

Throughout Aim 1, replicate standard curves were obtained on SAW Device 1 prior to each use to track changes in sensor performance with increasing sample loads.

Since the SAW sensor contains two independent parallel detectors with unique surface coatings, two simultaneous peaks were recorded each time a flavorant passed through the sensor. The height ratio of these simultaneous peaks is a consequence of the flavorant's relative affinity for each detector's surface coating and is different for D-limonene and methyl salicylate. This peak height ratio is independent of flavorant concentration and was used as a means of qualitatively identifying each flavorant.

### Aim 1: Determination of Dose-Response Relationships and Breath Kinetics for Sublingual Administration of Solutions Containing D-Limonene and Methyl Salicylate

Dose-response relationships were determined in four study participants following sublingual administration of solutions containing D-limonene and methyl salicylate. For each dose-response measurement, a 20 μL aliquot of aqueous ethanol containing a standardized amount of D-limonene, methyl salicylate and/or vanillin was placed under the tongue by the participant using an automatic pipettor and the mouth was closed. Five seconds after administration of the solution, the participant would blow a single five second breath into the SAW sensor. Subsequent breath samples were collected at 50, 95, 140 and 185 seconds after placement of the solution. The participant kept his or her mouth closed when not providing a sample and refrained from breathing through the mouth or talking during the collection period. Each participant repeated this protocol for the solutions shown in Table 2. In addition, the more promising solutions (7, 12 and 16) were tested in triplicate to estimate reproducibility within individuals.

TABLE 2
Composition of sublingual solutions tested in Aim 1.

| | Mass of flavorant in a | | |
|---|---|---|---|
| | 20 μL aliquot of solution (μg) | | |
| Solution ID | D-limonene | Methyl Salicylate | Vanillin |
| 1 | 0 | 0 | 0 |
| 2 | 0 | 0 | 30 |
| 3 | 1 | 0 | 30 |
| 4 | 3 | 0 | 30 |
| 5 | 10 | 0 | 30 |
| 6 | 30 | 0 | 30 |
| 7 | 100 | 0 | 30 |
| 8 | 300 | 0 | 30 |
| 9 | 0 | 1 | 30 |
| 10 | 0 | 3 | 30 |
| 11 | 0 | 10 | 30 |
| 12 | 0 | 30 | 30 |
| 13 | 0 | 100 | 30 |
| 14 | 0 | 300 | 30 |
| 15 | 30 | 10 | 30 |
| 16 | 100 | 30 | 30 |
| 17 | 300 | 100 | 30 |

Upon completion, results from Aim 1 were used to determine the proper doses of D-limonene and methyl salicylate needed for the powder feasibility studies in Aim 2. These feasibility studies and selection of the clinical study formulations are detailed below.

### Aim 2: Clinical Study Assessment of the SAW Sensor

The purpose of Aim 2 was to evaluate the detection of flavorants in the breath using four SAW devices to correctly identify five different SL placebo formulations (Table 3) following their administration in a cohort of eight study participants. At the start of each study visit, participants were randomly assigned one of the four SAW devices to collect and analyze breath samples. After a period of instruction on the use of the device, participants were randomly administered a series of five placebo SL formulations (Table 3) during the study visit.

TABLE 3
Composition of SL placebo formulations used in the clinical study.

| | Mass of flavorant in | | |
|---|---|---|---|
| Study | 30 mg aliquot of placebo SL matrix | | |
| Formulation ID | D-limonene | Methyl Salicylate | Vanillin |
| 1 | 0 | 0 | 0 |
| 2 | 0 | 0 | 30 |
| 3 | 0 | 30 | 30 |
| 4 | 200 | 0 | 30 |
| 5 | 200 | 30 | 30 |

A blinded researcher administered the study formulations as 30 mg powder aliquots contained in microcentrifuge tubes in a predetermined random order. To more accurately control the amount of D-limonene and methyl salicylate in the test powders, the flavorants were added immediately before giving the aliquot to the study participant. Formulations 1 and 2 were prepared and supplied by Westlab Pharmacy. Formulation 3 was prepared by adding a 1 μL aliquot of a USP grade ethanol solution containing 30 mg/mL of methyl salicylate to a 30 mg aliquot of the vanillin-containing placebo SL powder supplied by Westlab Pharmacy. Formulation 4 was prepared by adding a 1 μL aliquot of a 200 mg/mL solution of D-limonene in ethanol to the vanillin-containing powder, and formulation 5 was prepared by adding a 1 μL aliquot of an ethanol solution containing 30 mg/mL methyl salicylate and 200 mg/mL D-limonene to the vanillin-containing powder. The powder formulations were briefly stirred after the addition of the spiking solutions to distribute the flavorants throughout the powder and to ensure that no clumping of the powder had occurred. To maintain consistency, formulations 1 and 2 were “spiked” with μL aliquots of USP ethanol and mixed in a similar manner.

Before administration of each formulation, each study participant provided a 5 second baseline breath sample to be analyzed by the SAW device. Each subject was then instructed to place the powder formulation under the tongue, close his or her mouth, and allow the powder to dissolve for 15 seconds. The study participant then provided a second breath sample into the SAW device for analysis. The researcher verified that the breath samples were collected properly before administering the next formulation. A minimum washout time of 5-10 minutes was used between each formulation administration, which allowed for removal of the flavorants from the oral cavity. Each study participant completed three study visits on different days resulting in a total of 120 observations for Aim 2.

After completion of all study visits, the attending researcher compiled the raw sensor output and transferred it to a blinded researcher for interpretation. Since the raw data contained no information about the study participants or the formulations tested, the interpreting researcher used only the detector response to predict which formulation was administered to the study participant for a given device result. After using the SAW data to identify the formulations (120 analyses), the interpreting researcher submitted this assessment to the clinical research coordinator. The clinical research coordinator then released the randomization schedule to allow comparison between blinded SAW assessment and actual formulation use.

### Results and Discussion

Interaction of D-Limonene and Methyl Salicylate with the SAW Detectors and Stability of the SAW Devices in Aims 1 and 2.

The separation between D-limonene and methyl salicylate is produced by the SAW sensor's trap, which behaves like a small chromatographic column.

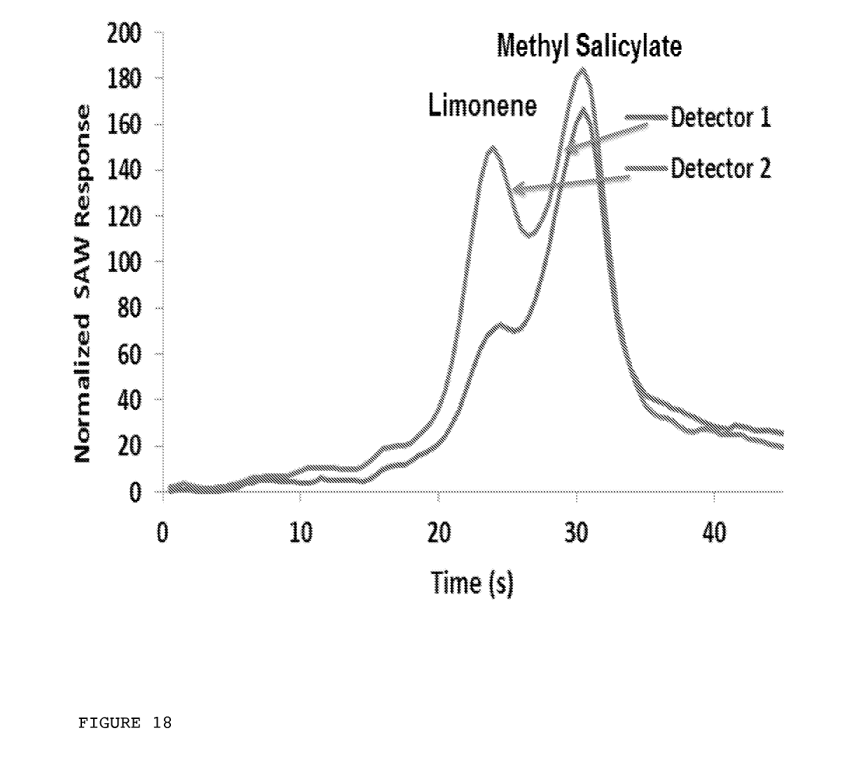

This separation can be quantified by measuring the resolution between the D-limonene peak and the methyl salicylate peak (FIG. 18). Resolution is defined as the difference in peak retention time divided by the average peak width. By this equation, a resolution of 1 would indicate completely resolved peaks. The SAW sensors separated D-limonene and methyl salicylate with a resolution of 0.5-0.6, which is sufficient for both qualitative discrimination and quantitative measurement of the two flavorants.

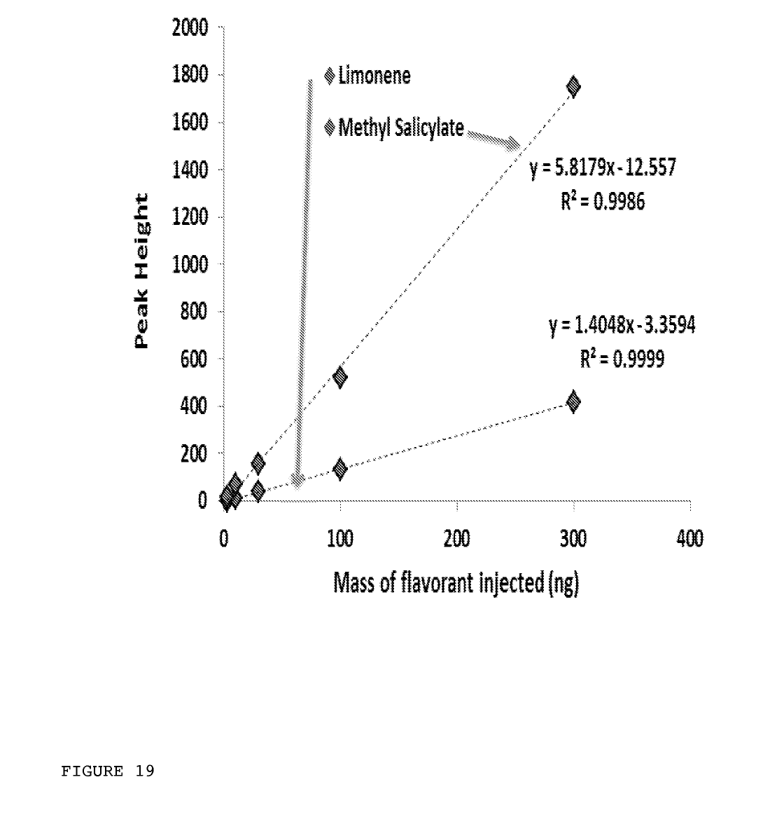

Methyl salicylate demonstrated a greater affinity toward both SAW detectors and produced a 3-5 times greater response than D-limonene for a given mass (FIG. 19). This resulted in a difference in the two flavorants' limits of detection. Only 3 ng of directly-injected methyl salicylate produced a quantifiable signal in the SAW sensor, whereas 10 ng of D-limonene was required for a comparable SAW signal (peak height).

Two effects contribute to this difference in sensitivity. First, methyl salicylate is less volatile than D-limonene and has a greater propensity to adhere to any surface at a given temperature. Second, methyl salicylate contains aromatic and ester functional groups that favor interaction with the polymer coatings on both detectors. D-limonene, in contrast, is a pure hydrocarbon and cannot exploit such molecular interactions. As a result, D-limonene produces a lower response in detector 1, which has a more hydrophilic surface than detector 2, whereas the response of methyl salicylate remains largely unchanged in either detector. This characteristic decrease of ˜50% in SAW response between detectors 1 and 2 for D-limonene was consistent across all SAW sensors and was useful for qualitative identification of D-limonene.

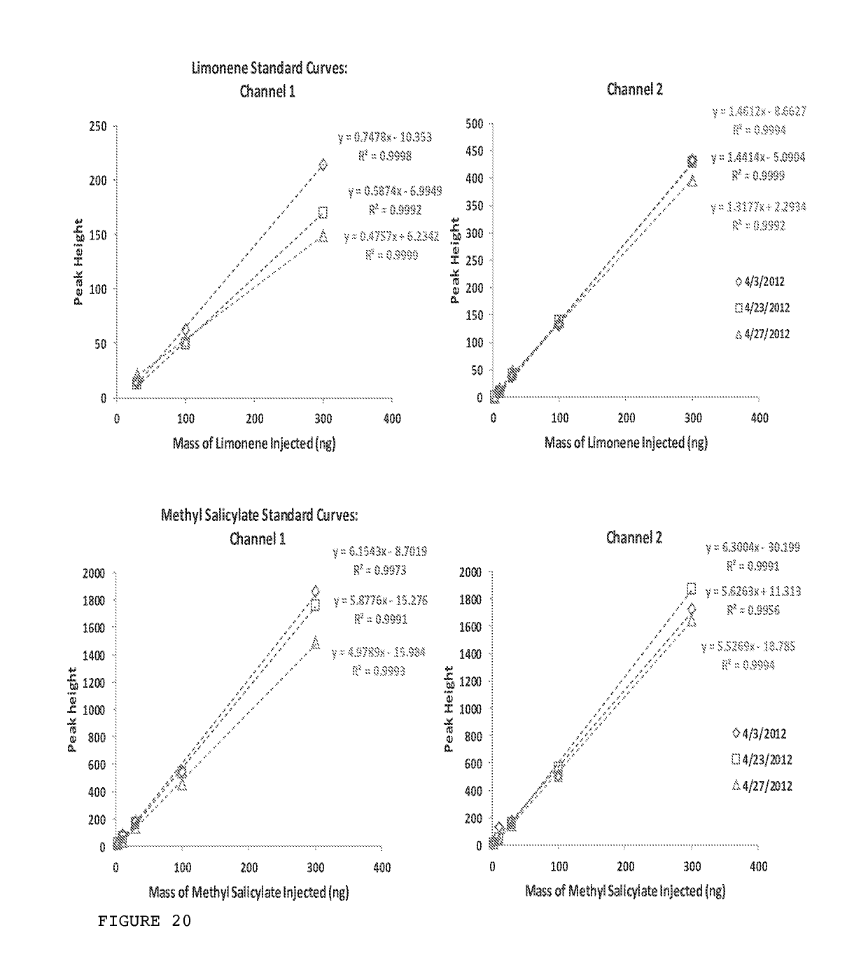

The SAW devices displayed highly linear relationships between detector response and mass of directly-injected D-limonene or methyl salicylate over a range of 3 to 300 ng (FIG. 20). SAW device 1 was the only instrument used during Aim 1 and processed ≈250 breath samples over one month. The linearity of response for both detectors was unchanged throughout this usage but some decrease in sensitivity was observed. Although the lowest concentration standards remained detectable, the sensitivity of detector 1 decreased by 36% and 20% for D-limonene and methyl salicylate, respectively. The sensitivity of detector 2 was less affected by use and decreased by only 8% and 12% for D-limonene and methyl salicylate, respectively. These results are encouraging considering that the number of samples analyzed in Aim 1 was roughly equivalent to one year of once-daily use and these losses did not impact the ability of the SAW device to accurately detect flavorants in the breath throughout the study in Aim 2.

Differences in sensitivity were observed among the SAW devices. Although SAW device 1 was still performing well by the end of Aim 1, it was the least sensitive of the four units. As compared to device 1, device 4 was routinely 15-30% more sensitive, device 3 was 50-100% more sensitive, and device 2 was over 200% more sensitive.

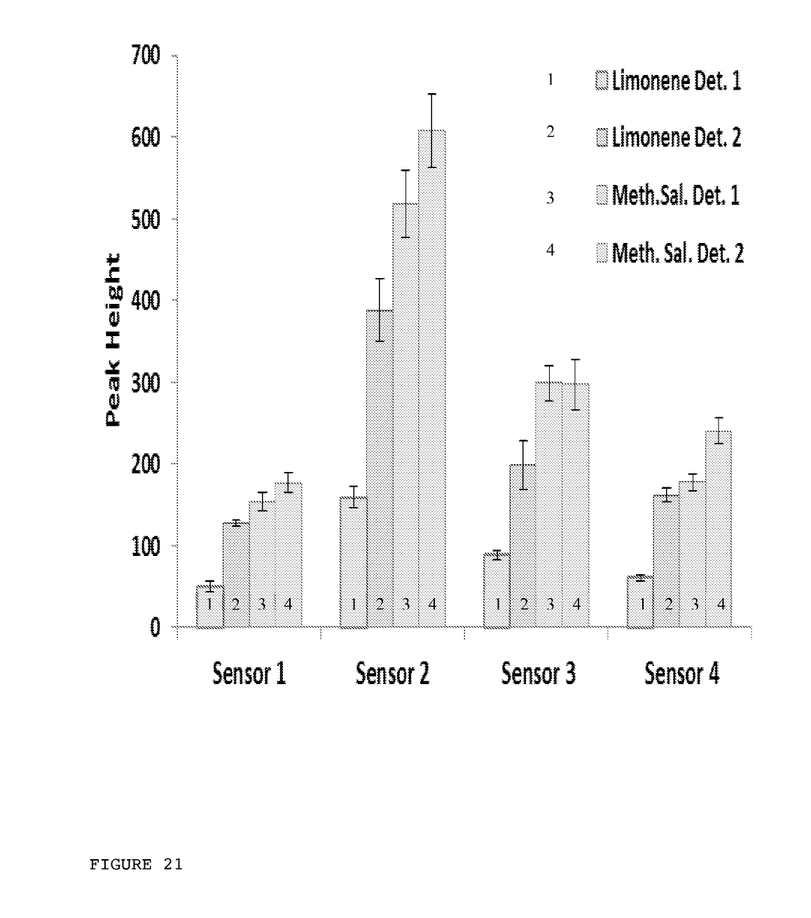

Despite these differences in absolute response, all of the devices performed consistently during the clinical study (FIG. 21). Compared to Aim 1, the SAW units processed a significantly lower number of breath samples in Aim 2 and, correspondingly, experienced a substantially smaller change in sensitivity over the course of the study. The coefficient of variability in check standards analyzed on the devices during use was typically under 10% and only rose to 15% for D-limonene analyzed on SAW device 3.

### Breath Kinetics and Dose-Response Relationships for Orally-Administered Flavorants.

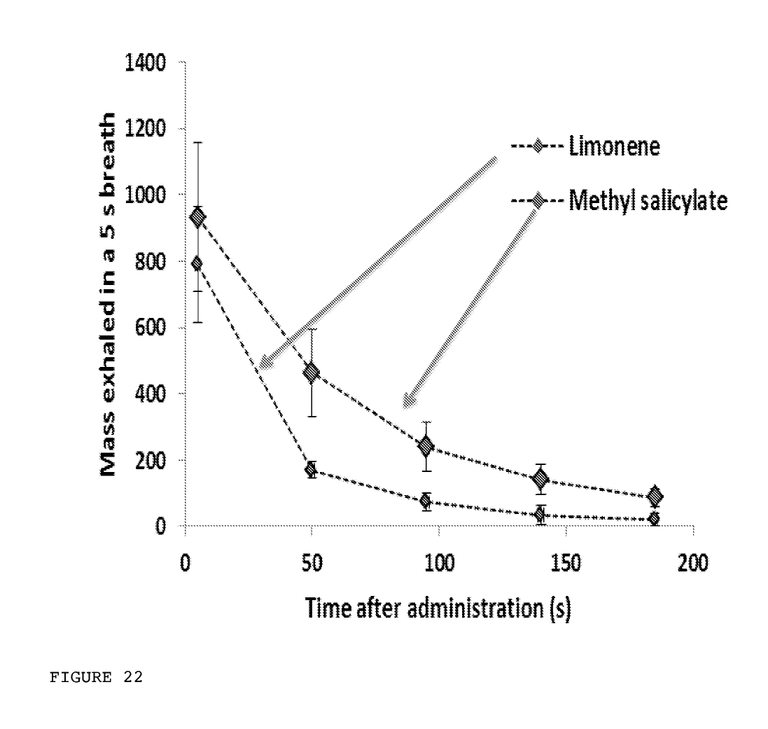

As shown in FIG. 22, the concentration of flavorants appearing in exhaled breath decayed exponentially over time for both D-limonene and methyl salicylate after a single SL administration.

As expected, the more volatile D-limonene was depleted at a faster rate than methyl salicylate. Since the SAW devices sample a consistent volume, the mass collected per breath sample is proportional to concentration. Therefore, on average, 78% of the D-limonene breath concentration measured at 5 seconds was lost by 50 seconds, whereas 50% of the methyl salicylate breath concentration was lost at the same time. These results suggest that breath samples should be taken as soon as practically possible for a SL tablet containing D-limonene, and sampling within one minute after administration may be necessary for more volatile flavorants.

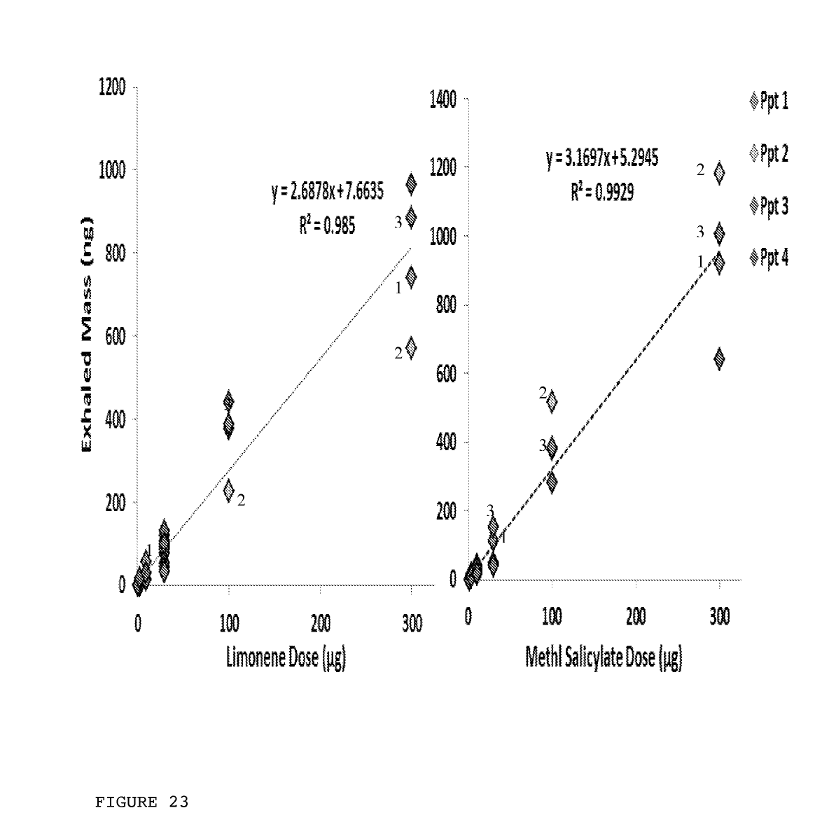

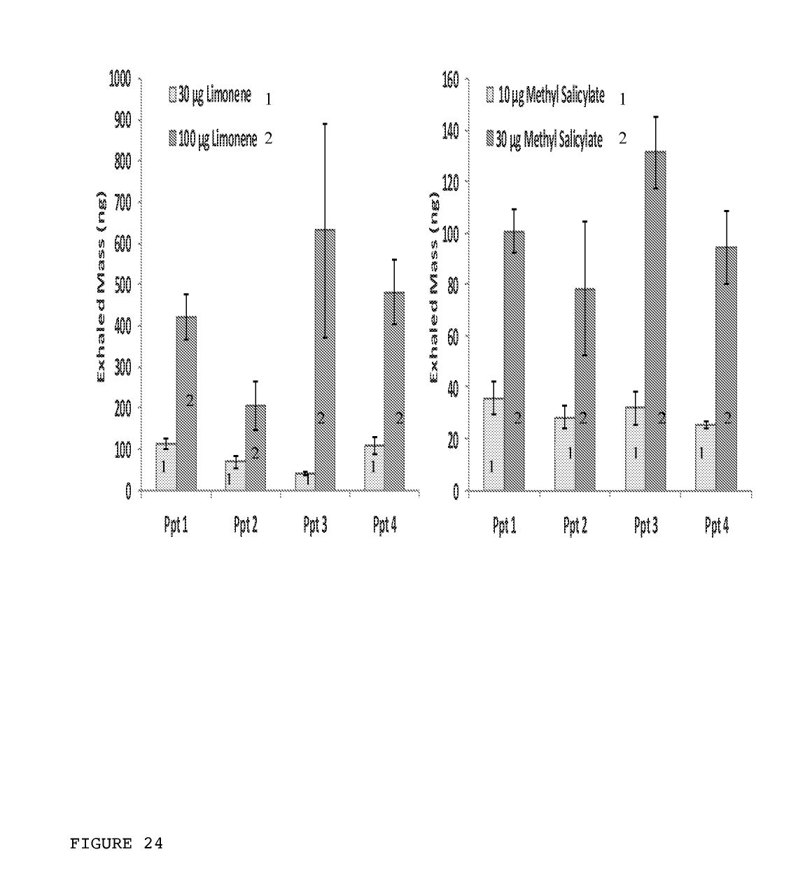

Both flavorants displayed a linear relationship between administered dose and exhaled mass (FIG. 23). There was significant inter-individual variability for both flavorants; however, replicate doses of methyl salicylate produced a more consistent breath concentration among replicates and across study participants than did D-limonene (FIG. 24). For D-limonene, the variability in exhaled breath concentration was generally greater among study participants than among replicates. This finding suggests that individual variability may influence exhaled breath concentrations as much as flavorant mass within the SL powder.

An important design consideration moving forward will be determining what the range of this variability is and how powder doses will need to be modified to compensate, but those skilled in the art are well placed to determine optimium conditions based on the present disclosure and routine experimentation.

### Initial Powder Formulations: Stability and Feasibility Studies.

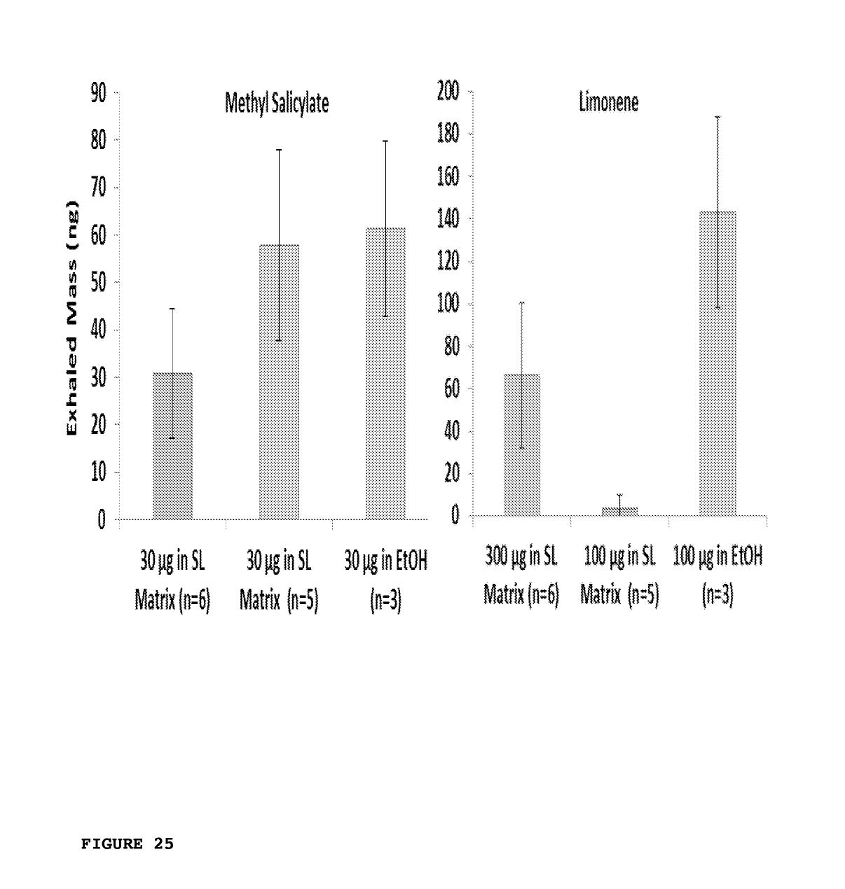

After an initial assessment of the results from Aim 1, it was predicted that a D-limonene mass of 100 μg and a methyl salicylate mass of 30 μg per 30 mg dose of placebo SL formulation were the proper doses to use for the clinical study in Aim 2. A 3 g batch of placebo SL powder was prepared using this composition, and several 30 mg aliquots of this powder were tested sublingually (FIG. 25). While the methyl salicylate performed as expected, D-limonene was barely detectable. The D-limonene concentration in the batch was increased to 300 μg per 30 mg powder, and the batch was retested along with sublingual doses of 100 μg D-limonene and 30 μg methyl salicylate dissolved in ethanol.

A 30 μg aliquot of methyl salicylate produced comparable breath responses when administered in powder or in solution, but D-limonene was lost preferentially from the SL powder.

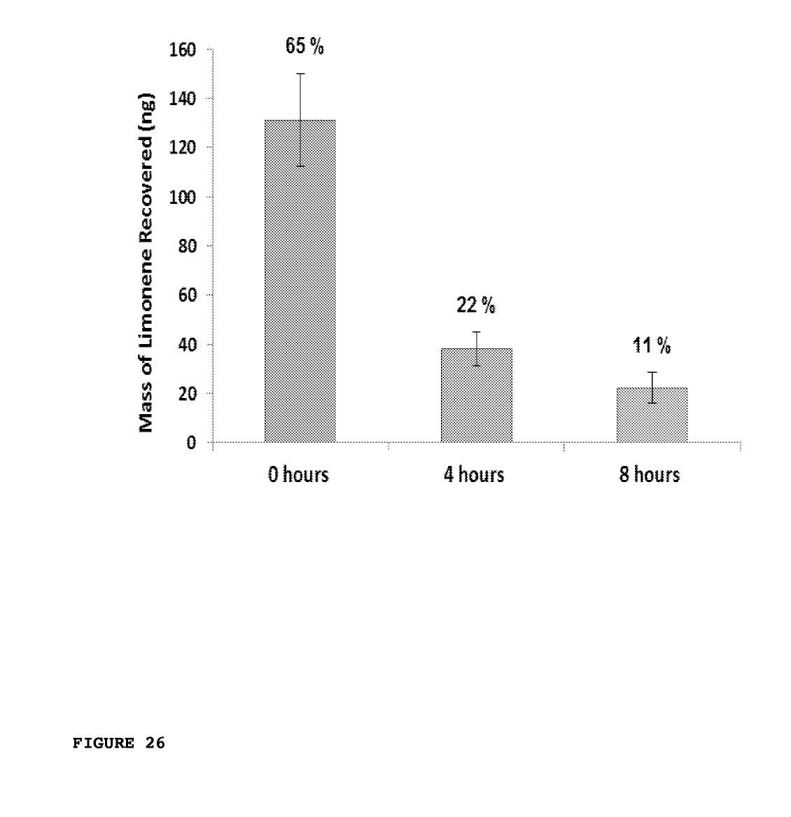

The principal cause of this loss was confirmed by preparing a fresh 3 g batch of placebo SL powder with D-limonene and methyl salicylate. Three replicate 30 mg portions of this powder were removed immediately after preparation and extracted with acetonitrile to recover the D-limonene. The extracts were analyzed by liquid chromatography-mass spectrometry (LCMS). This was repeated with three more portions after four hours and again after eight hours (FIG. 26).

Approximately 65% of the D-limonene dose was recovered from the powder just after preparation. At four hours, only 22% of the expected dose was recovered, and this fell to 11% at eight hours. It was clear from these results that neat D-limonene was lost rapidly from the powder surface. To account for this rapid loss, it was decided that the D-limonene would be added to the individual doses of placebo SL powder just before administration to the participants for the clinical study. A series of formulations were prepared in this manner by adding 200 μg D-limonene and 30 μg methyl salicylate to 30 mg portions of the SL matrix. These formulations were tested in four participants and found to produce easily detectable breath signals for both flavorants.

### Results of the Double Blind Clinical Study (Aim 2).

Four males and four females were recruited for the clinical study. Ages ranged from 18-70 years with a mean age of 32 years. Study participants were non-smokers and free from any reported respiratory ailments.

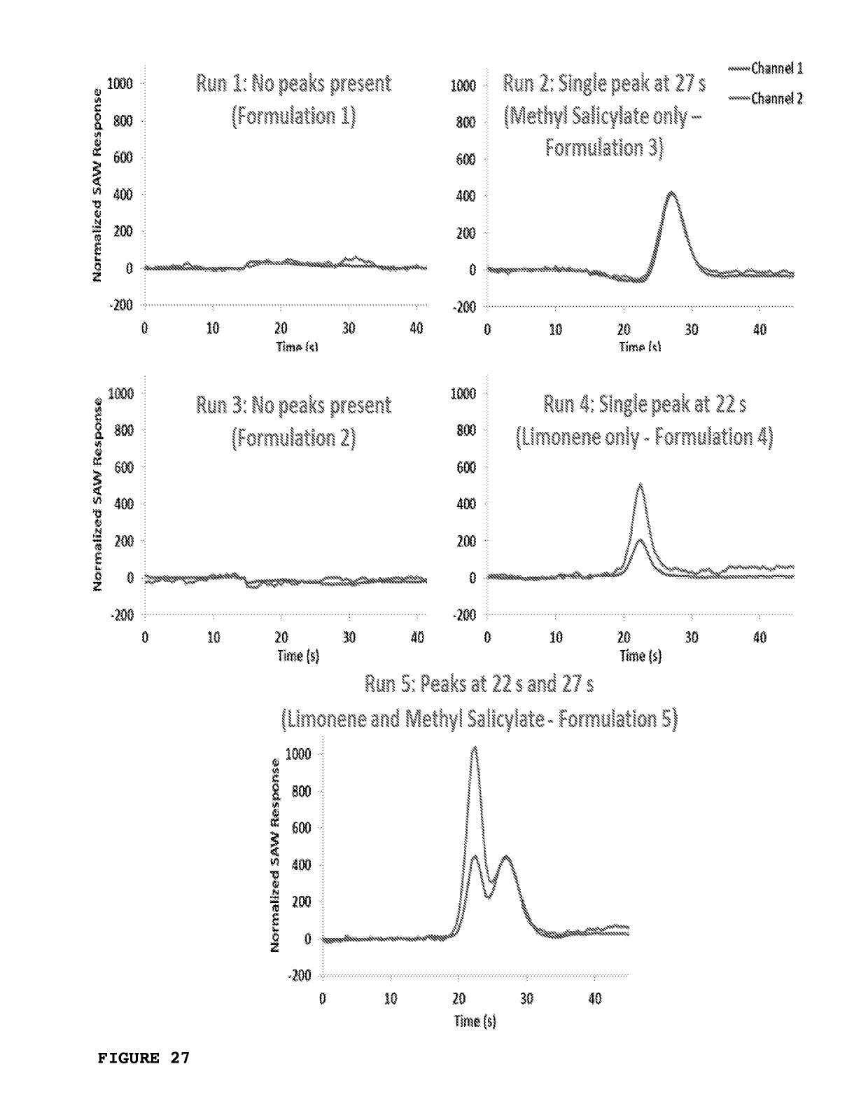

All study participants quickly learned to use the device. They also required very little practice to become adept at taking the SL formulations and providing breath samples with the proper timing. Consequently, over the course of the study, there were no errors resulting from improper breath sampling, incorrect placement of the formulation, or coordination of these two actions. FIG. 27 illustrates the typical SAW responses for the five SL formulations that were randomly administered to a particular study participant during one of the study visits (SAW009 on visit 2).

The general sequence of events for a complete run was as follows: 1) collect an initial breath sample, 2) administer the SL formulation, and 3) collect another breath sample. This entire process took ≈90 seconds for both breath and data collection, but the two breath collections were completed in 50 seconds. The initial breath sample was taken primarily to ensure that no carry-over had occurred from a previous sample, but it was also used to enhance the sensitivity of the sample breath by providing a baseline with which to subtract out potential interferences and other artifacts. Study participants were compliant with the request to refrain from eating, drinking, or chewing gum to reduce the likelihood of interferences. No breath-based interferences were noted in this study, but this does not preclude the possibility of observing them in a larger population. Taking a baseline breath sample did not add much time to the overall process, and subtracting the baseline breath results from the sample breath results simplified the output data and allowed the creation of an automatic algorithm for use in interpreting the results. Future work will explore what algorithms could be used for this purpose and how effective they are relative to a human observer.

In every case (120/120 experiments), the blinded researcher was able to correctly identify when the formulations containing D-limonene and/or methyl salicylate were administered and could distinguish these formulations from those that did not contain flavorants or contained only vanillin (Table 4). Each SAW device performed equally well with regard to this qualitative assessment.

TABLE 4
Comparison between the predicted dosing schedule (obtained by a blinded researcher using SAW data) and the actual dosing schedule.
Breath samples that contained no identifiable D-limonene or methyl salicylate were designated “B” by the researcher (Note: Since
formalations 1 and 2 lacked flavorants that were detectable by the SAW sensor, they were indistinguishable
by the blinded researcher and would both be given a designation of “B”.).

| | | First | | Second | | Third | | Fourth | | Fifth | |
|---|---|---|---|---|---|---|---|---|---|---|---|
| | | Formulation | | Formulation | | Formulation | | Formulation | | Formulation | |
| | | Blinded | | Blinded | | Blinded | | Blinded | | Blinded | |
| Participant | Visit | Assessment | Actual | Assessment | Actual | Assessment | Actual | Assessment | Actual | Assessment | Actual |
| SAW005 | 1 | 4 | 4 | 5 | 5 | B | 2 | B | 1 | 3 | 3 |
| SAW006 | 1 | B | 2 | 3 | 3 | 5 | 5 | B | 1 | 4 | 4 |
| SAW007 | 1 | 5 | 5 | 4 | 4 | B | 1 | 3 | 3 | B | 2 |
| SAW008 | 1 | 4 | 4 | B | 2 | 3 | 3 | 5 | 5 | B | 1 |
| SAW009 | 1 | B | 1 | 4 | 4 | 3 | 3 | B | 2 | 5 | 5 |
| SAW010 | 1 | B | 2 | 5 | 5 | B | 1 | 4 | 4 | 3 | 3 |
| SAW011 | 1 | 4 | 4 | B | 2 | 3 | 3 | 5 | 5 | B | 1 |
| SAW012 | 1 | 3 | 3 | 4 | 4 | B | 2 | B | 1 | 5 | 5 |
| SAW005 | 2 | 4 | 4 | B | 1 | 3 | 3 | 5 | 5 | B | 2 |
| SAW006 | 2 | 3 | 3 | 5 | 5 | 4 | 4 | B | 1 | B | 2 |
| SAW007 | 2 | 3 | 3 | B | 1 | B | 2 | 4 | 4 | 5 | 5 |
| SAW008 | 2 | 3 | 3 | 5 | 5 | 4 | 4 | B | 2 | B | 1 |
| SAW009 | 2 | B | 1 | 3 | 3 | B | 2 | 4 | 4 | 5 | 5 |
| SAW010 | 2 | B | 2 | B | 1 | 3 | 3 | 4 | 4 | 5 | 5 |
| SAW011 | 2 | 5 | 5 | B | 2 | 4 | 4 | B | 1 | B | 2 |
| SAW012 | 2 | B | 1 | B | 2 | 3 | 3 | 5 | 5 | 4 | 4 |
| SAW005 | 3 | 4 | 4 | B | 1 | 5 | 5 | 3 | 3 | B | 2 |
| SAW006 | 3 | 3 | 3 | B | 1 | B | 2 | 4 | 4 | 5 | 5 |
| SAW007 | 3 | B | 1 | 5 | 5 | 4 | 4 | B | 2 | 3 | 3 |
| SAW008 | 3 | 4 | 4 | B | 2 | 5 | 5 | 3 | 3 | B | 1 |
| SAW009 | 3 | B | 1 | B | 2 | 3 | 3 | 4 | 4 | 5 | 5 |
| SAW010 | 3 | 4 | 4 | 3 | 3 | B | 1 | 5 | 5 | B | 2 |
| SAW011 | 3 | 5 | 5 | 4 | 4 | 2 | 2 | B | 1 | 3 | 3 |
| SAW012 | 3 | B | 1 | 4 | 4 | 5 | 5 | 3 | 3 | B | 2 |

By designating samples containing D-limonene or methyl salicylate as “positive” and samples without the more volatile flavorants as “negative”, this data can be analyzed using standard binary classification tests:
Number of observations=120
Number of true positives (TP, formulations 3, 4 and 5)=70
Number of true negatives (TN, formulations 1 and 2)=50
Number of false positives (FP)=0
Number of false negatives (FN)=0

Sensitivity=TP/(TP+FN)×100=100%

Specificity=TN/(TN+FP)×100=100%

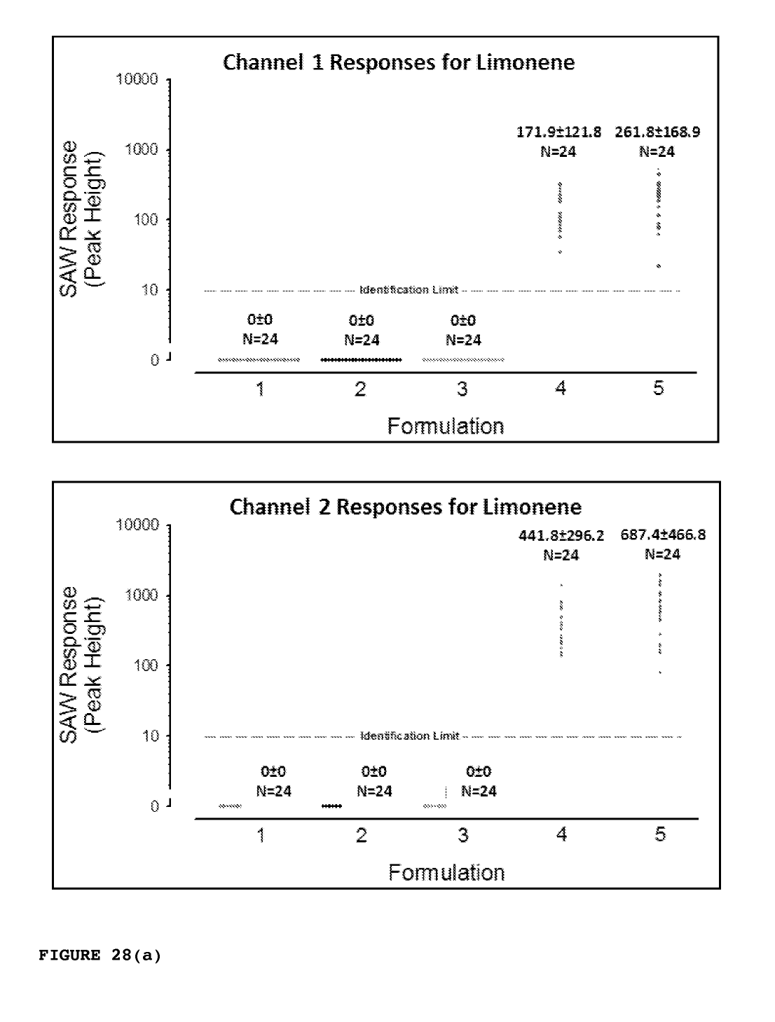

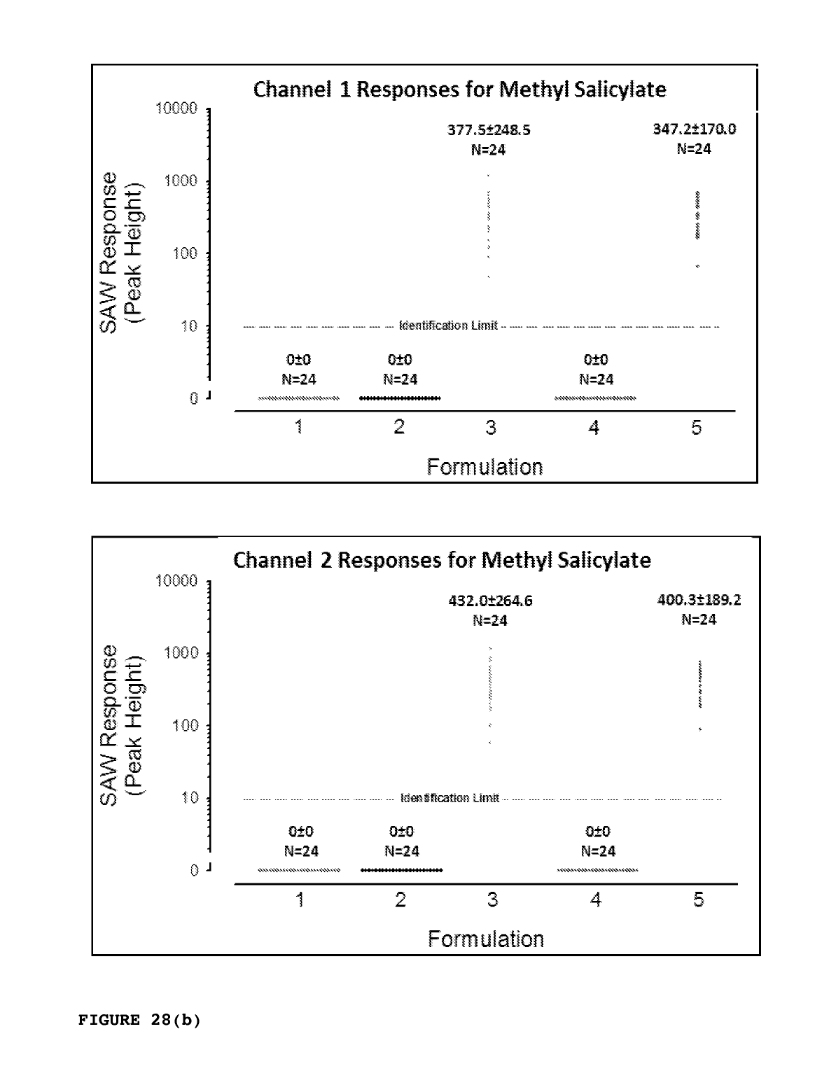

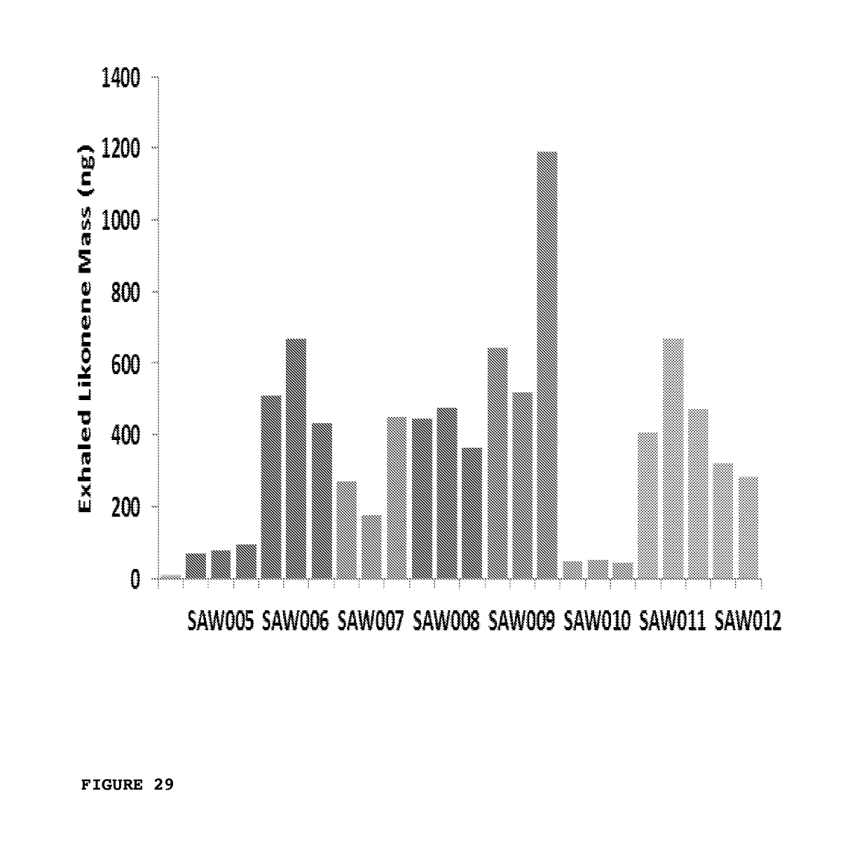

From a qualitative standpoint, the SAW devices performed well and as expected. Despite administering the same doses of each flavorant, there was a much larger variability in measured peak heights for D-limonene and methyl salicylate in breath samples collected in Aim 2 than for breath samples collected in Aim 1. Peak heights for both flavorants varied by 10 to 20 fold across formulations 3, 4 and 5 (FIG. 28). Three key factors contribute to this variability in measured peak heights. First, the SAW devices have inherent differences in sensitivity. Second, release rates of flavorants depend on the manner in which the placebo SL powders were prepared. Since it was difficult to create uniformly homogeneous mixtures when spiking with such small individual doses, it is not surprising that this would have an effect on the release rates of the flavorants from the powder and the resulting breath concentrations. Finally, as with the Aim 1 studies, inter-individual differences played a role. This is easily illustrated by converting the breath responses for D-limonene measured in all replicates of formulation 5 to exhaled mass and plotting them by study participant (FIG. 29).

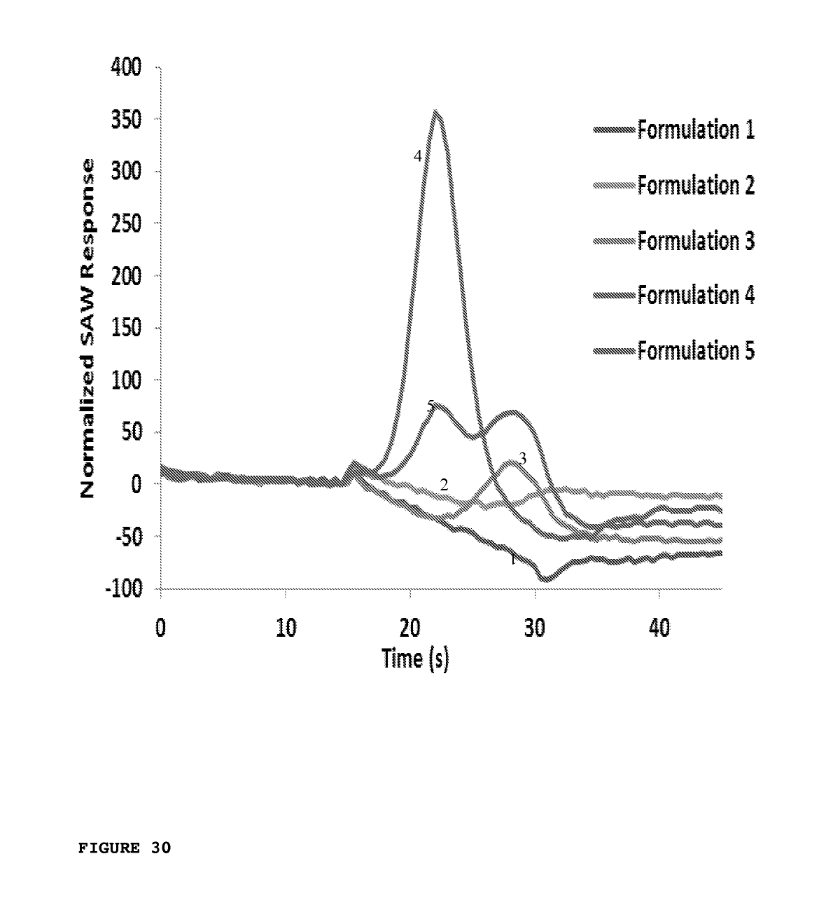

Study participant SAWO10 typically produced lower breath levels of D-limonene and methyl salicylate than the other study participants and generated the lowest response for a set of SL formulations. However, it was encouraging that even the weakest response was 5 times the peak height needed to identify the presence of D-limonene (FIG. 30).

### CONCLUSIONS

GRAS flavorants can be successfully used as adherence markers for SL tablets. The four SAW devices tested in this study reliably detected D-limonene and methyl salicylate in exhaled breath following the administration of placebo SL powders containing only microgram quantities of either flavorant. The SAW devices were used by multiple study participants and were able to distinguish between SL formulations containing one or both flavorants. The SAW sensors could routinely detect 10 ng of D-limonene and 3 ng of methyl salicylate and maintained this sensitivity over the course of the studies.

The formulations tested in this study represent “proof of concept” SMART™ SL formulations. Simple SL formulations of “A”, “B”, and “A+B” types were easily prepared and readily distinguished using the SAW devices in the clinical study setting. By using amounts (and combinations) of these flavorants which are far below the levels seen in typical foodstuffs (e.g., gum and candy), the formulations evaluated in this clinical study produced breathprints (i.e., patterns and concentrations) of D-limonene and methyl salicylate that would be difficult to reproduce. The flavorants could also be used in such small quantities that the patient would not be able to taste the flavor, discern what flavoring agent was used, or discriminate among multiple flavors. Given the rapid dissolution of SL medications in the mouth, these types of flavorants could be used to provide a measure of definitive adherence. For example, it is unlikely that the levels of methyl salicylate and D-limonene produced in the breath simultaneously following administration of formulation number 5 could be reproduced without some degree of sophistication. However, the quantitative nature of the SAW sensor suggests that, if needed, more complex and more characteristic combinations of flavorants are entirely possible.

The challenge in terms of creating a SAW-based SMART™ Adherence System to detect SL tablet ingestion appears to be at the level of creating an optimized SL formulation that stably incorporates the flavorants. Methyl salicylate displays greater stability in the SL powder than D-limonene, even when it is simply mixed in as a neat liquid. SMART™ formulations using only one flavorant are possible, but since absolute breath response will be used to distinguish the SL tablet from other potential sources of the flavorant, inter-individual and inter-occasion variability will be more of an issue. It is likely that pharmaceutical formulators can readily address these issues. The uniformity and quality of a GMP SL formulation containing these types of flavorants will likely reduce variability. The advantage of a SL formulation containing two flavorants is that a relative ratio of the SAW responses to the two flavorants can be used instead of absolute SAW responses, which makes a two-flavorant system preferable.

In summary, we conclude that the use of the SAW-based SMART™ Adherence System to detect flavorants in the breath following ingestion of SL tablets containing selected flavorants shows significant promise to provide a definitive assessment of medication adherence in clinical trial and disease management settings.

### Example 2

### Real Time High Resolution Mass Spectrometry of Exhaled Flavorants Following Administration of Lemon, Root Beer and Wintergreen Flavored Pharmaceutical Powders from FONA International to the Surface of the Tongue

### Instrumentation and Methods:

High resolution spectra for flavor headspace standards and breath samples were analyzed using a ThermScientific Orbitrap LCMS operating in atmospheric pressure ionization mode (API) and scanning from 100-200 dalton. The API source has been modified to allow the direct introduction of volatile samples.

To obtain qualitative API spectra for limonene, carvone, methyl salicylate and menthol, 30 mL samples of the headspace from sample bottles containing neat flavor compounds were collected in a glass syringe and injected into the API source.

API spectra for components appearing in the breath were obtained by blowing a single 5 s breath directly into the API source. These breath samples were obtained 30 s after placing the test powder onto the tongue and ˜10-15 s after dissolution of the powder.

For the lemon and root beer flavored powders, 100 mg of powder was administered prior to taking a breath sample. For the wintergreen powder, 20 mg of powder was taken.

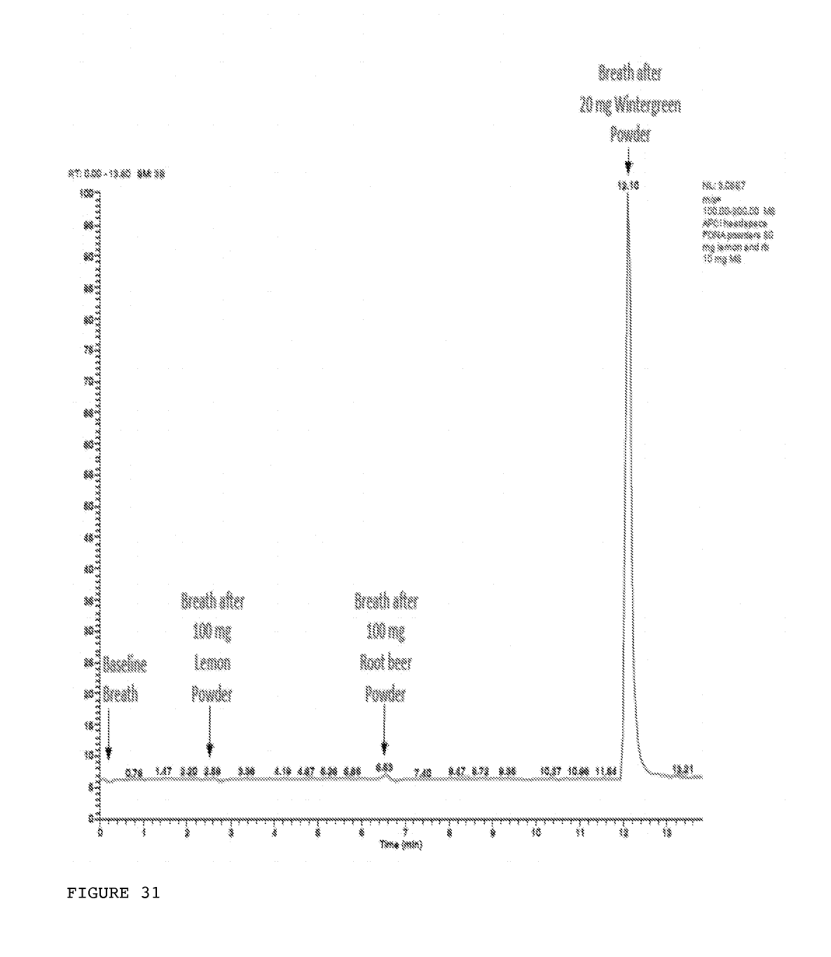

FIG. 31—Total Ion Chromatograph (TIC) of baseline breath sample and breath samples collected after administration of the FONA powders. The TIC is a sum of all ions measured during the breath sample. As a result, peak size roughly corresponds to the mass of volatile components present. Very little volatile material is observed in the baseline breath sample and after the administration of either lemon or root beer powders. In contrast, the wintergreen powder releases a large amount of volatile material.

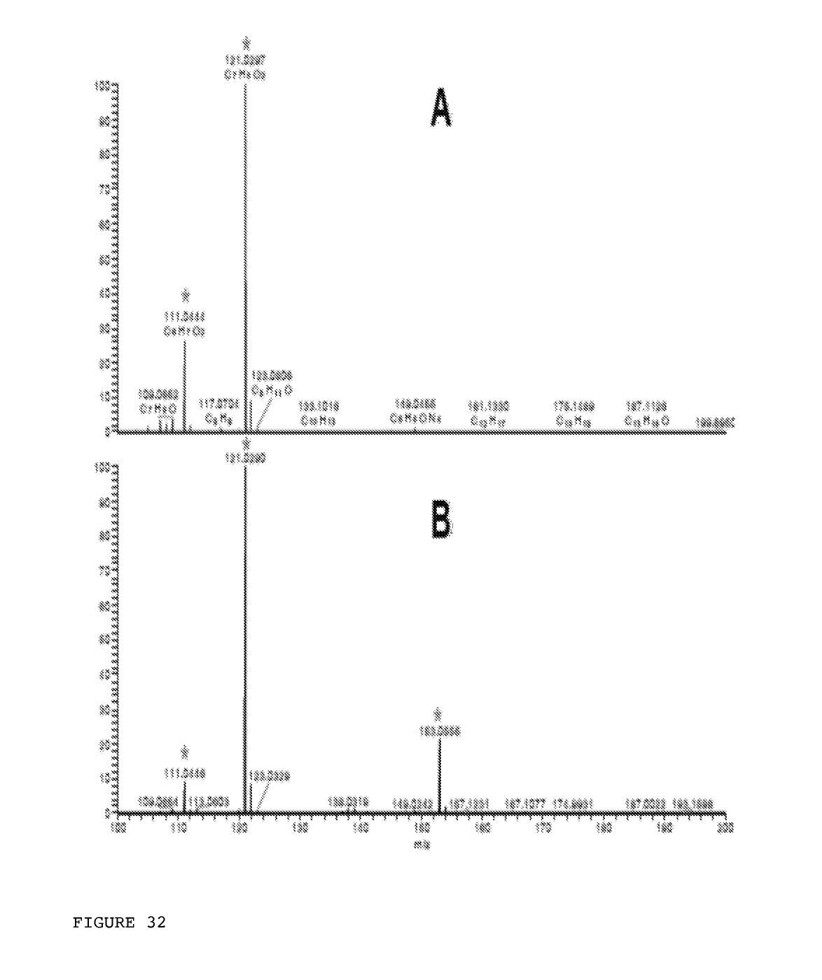

FIG. 32—High resolution API mass spectra of methyl salicylate (A) and the breath sample following administration of the wintergreen powder (B): All of the abundant masses present in the TIC of the wintergreen powder breath sample are produced by the fragmentation of methyl salicylate marked with an (*). Even the additional mass at 153 in the breath sample is due to protonated and unfragmented methyl salicylate, which becomes more prominent in the presence of higher breath humidity. The large breath response following the FONA wintergreen sample is due to methyl salicylate.

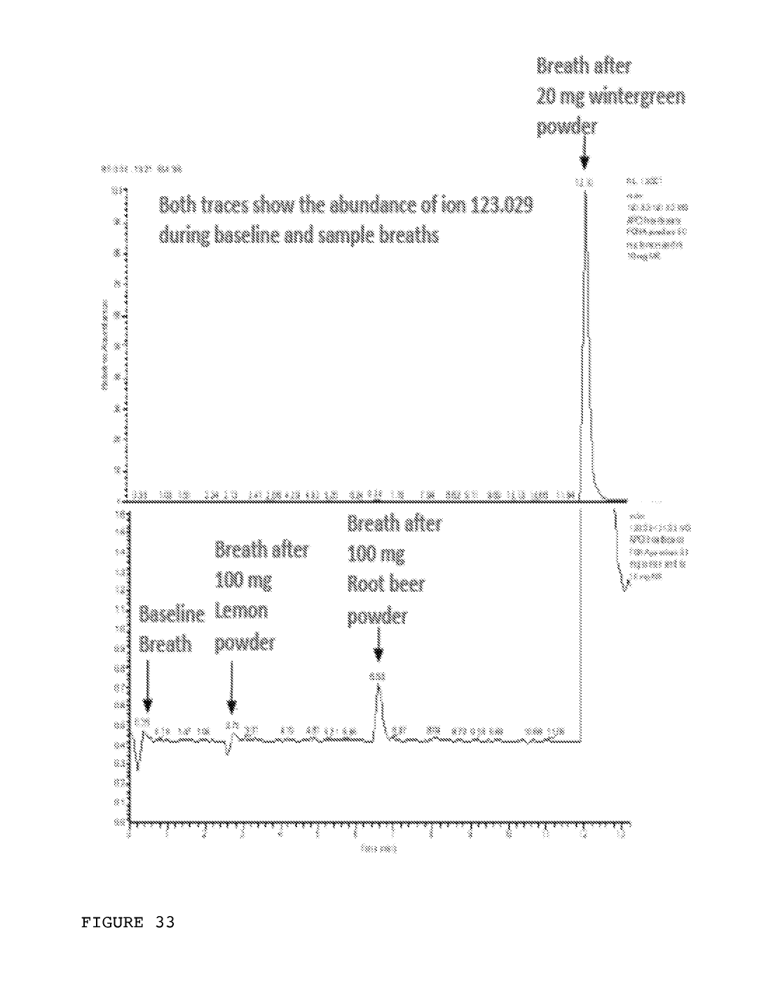

FIG. 33—High resolution selected ion (SI) chromatograms of the baseline breath samples and breath samples collected after FONA powder administration: By selecting a high resolution mass fragment that is characteristic for methyl salicylate (123.029 dalton), a more sensitive analysis of the breath samples is possible. The top trace shows the full scale SI chromatogram for the breath samples. The bottom trace shows the same chromatogram with the y-axis expanded ˜50 x. No methyl salicylate is seen in the lemon powder, but a small amount is present in the root beer flavoring. FONA wintergreen powder contains 500-1000 times more methyl salicylate than the root beer flavoring. No other volatile flavorings (limonene, menthol or carvone) were detected in the samples.

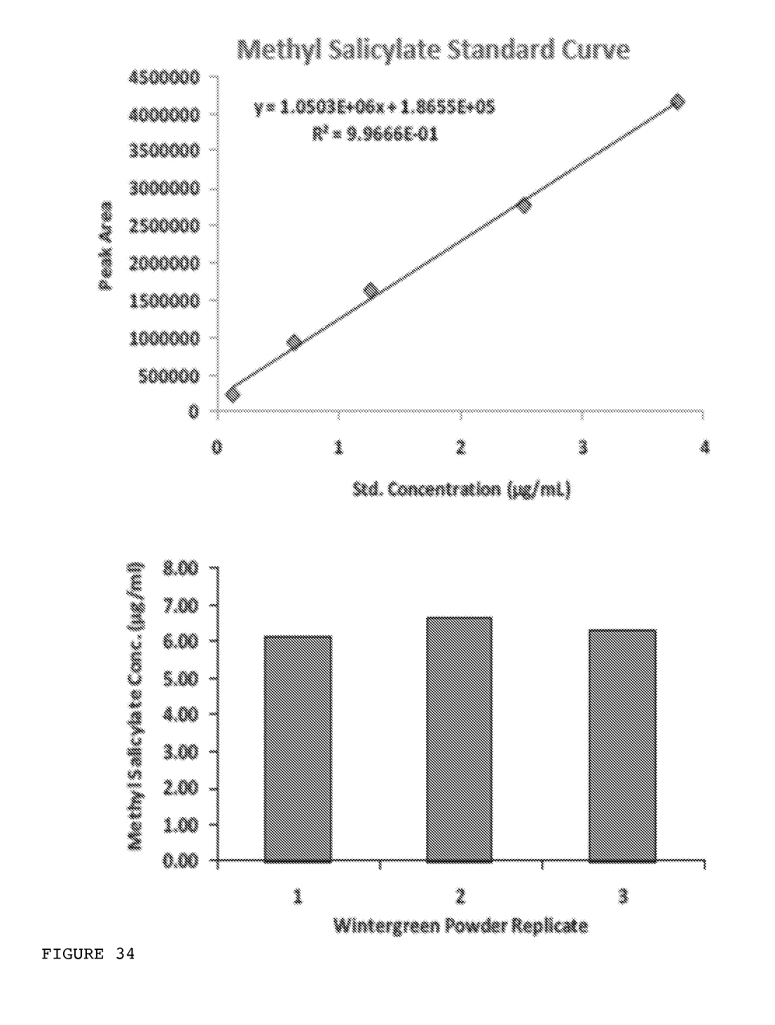

FIG. 34—Concentration of methyl salicylate in the FONA powders: Three replicate 50 mg portions of each FONA powder were extracted overnight (˜18 hours) with methanol to isolate the methyl salicylate. Aliquots of the lemon and root beer powder extracts were diluted 50 fold with water and analyzed by LCMS. Aliquots of the wintergreen powder extracts were diluted 133 times and analyzed by LCMS. The methyl salicylate concentration was below the limit of detection in the lemon and root beer powder extracts. This translates to less than 0.25 μg of methyl salicylate per mg of powder. In contrast, the wintergreen powders contained an average of 6.40 μg of methyl salicylate per mg of powder or 320 μg per 50 mg dose.

| | | Meth. Sal. | Meth. Sal. | Methyl |
|---|---|---|---|---|
| | | Peak | Conc. In | Salicylate |
| Wintergreen | Replicate | Area in | Diluted | Conc. |
| Powder | Mass | Diluted | Extract | in powder |
| Replicate | (mg) | Extract | (μg/mL) | (μg/mg) |
| 1 | 49.54 | 2605162 | 2.30 | 6.18 |
| 2 | 53.89 | 3028198 | 2.71 | 6.68 |
| 3 | 53.09 | 2847085 | 2.53 | 6.35 |
| | | | Average = | 6.40 |
| | | | CV = | 3.94 |

### Example 3

### GCMS Analysis and SAW Detection of Flavorants in Alayert™ ODTs and USP Grade Wintergreen Flavored Pharmaceutical Powder from FONA

### Section 1: Initial GCMS Analysis of the Test Materials

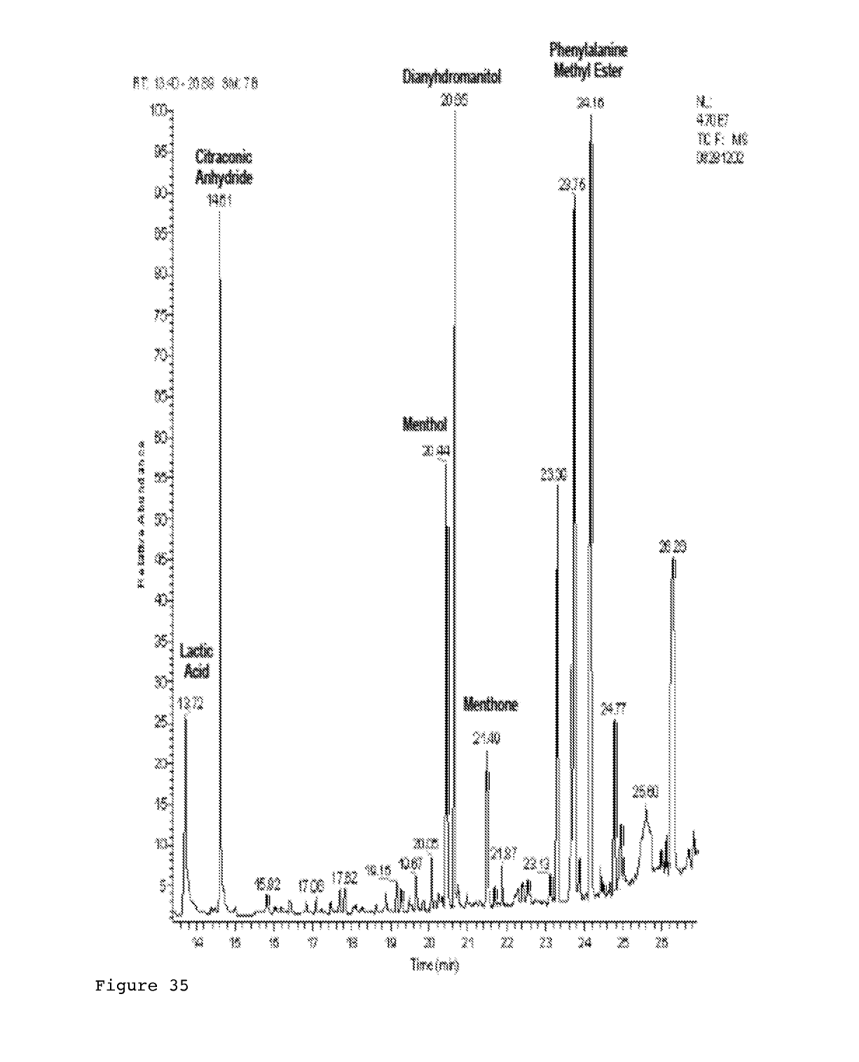

FIG. 35: GC/MS Analysis of ALAVERT Fresh Mint Tablet (300 mg Tablet containing 10 mg Loratadine). Menthol was the most abundant SAW-detectable component observed in the Fresh Mint formulation by the GCMS. We are currently working on quantitating the amount of menthol present in a single tablet.

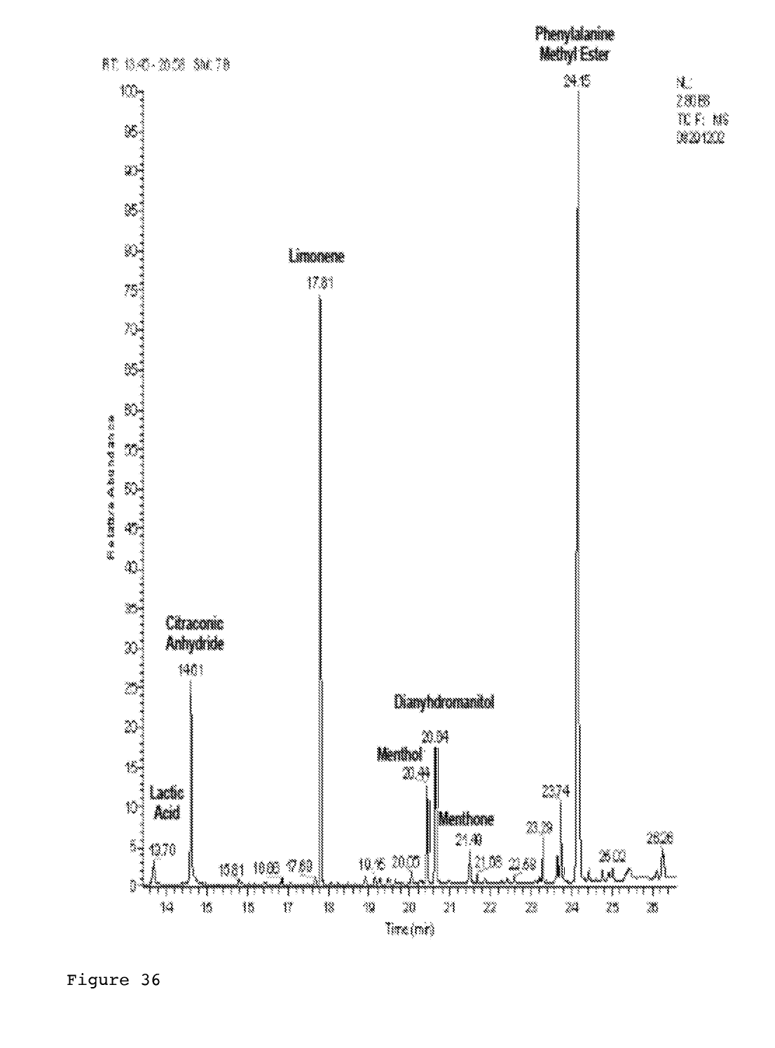

FIG. 36: GC/MS Analysis of ALAVERT Citrus Blast Tablet (300 mg tablet containing 10 mg Loratadine). Limonene was the most abundant SAW-detectable flavorant observed in the Citrus Blast formulation. A single 300 mg tablet tablet contained 162 μg of limonene.

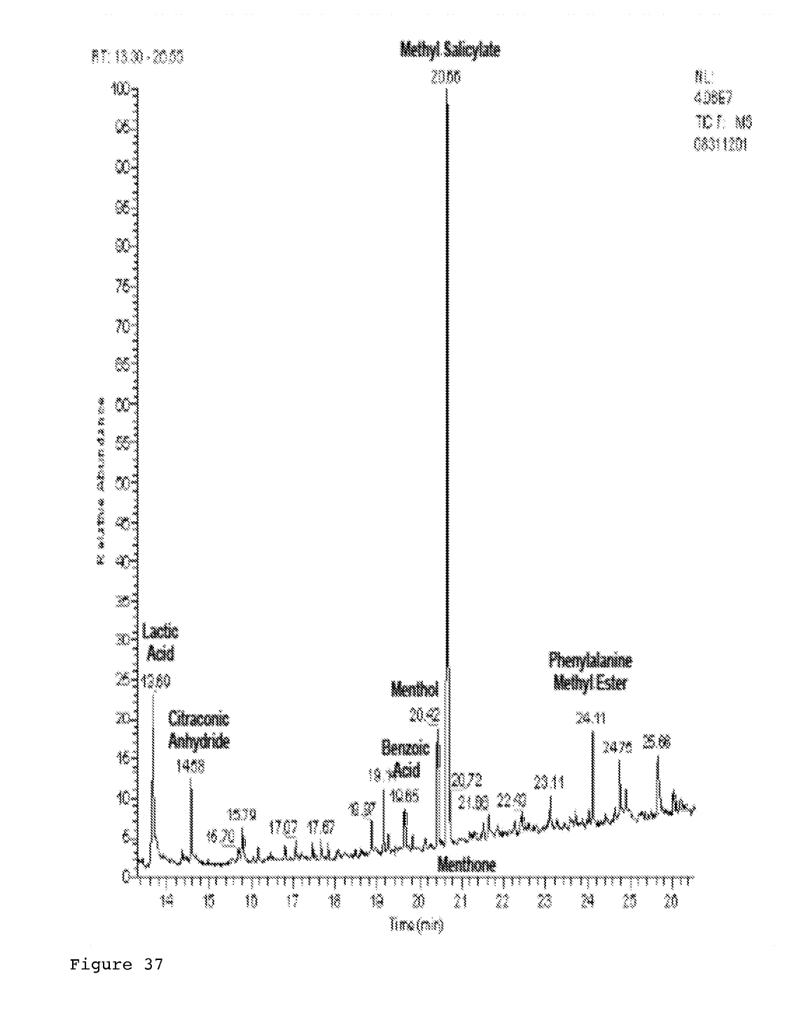

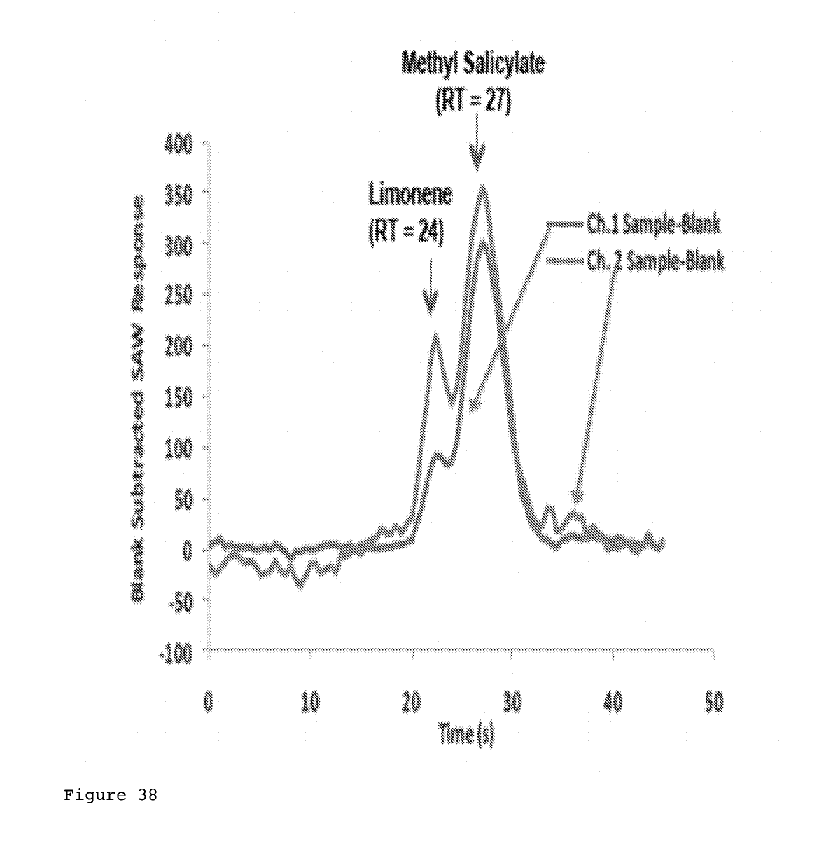

FIG. 37: GC/MS Analysis of Wintergreen Flavor (FONA). Methyl salicylate was the most abundant SAW-detectable flavorant observed in the FONA Wintergreen flavoring powder. Initial quantitation of the methyl salicylate concentration is being confirmed.

### Section 2: Qualitative SAW Analysis of Reference Standards

FIG. 38: SAW Reference Standards—100 ng of limonene and 30 ng methyl salicylate injected directly into the device. The chromatogram shown was obtained today (Aug. 31, 2012) using one of the SAW devices from the recently completed sublingual tablet clinical trial (Unit 1111-02-B). All SAW reference standard and breath sample data were collected using this unit.

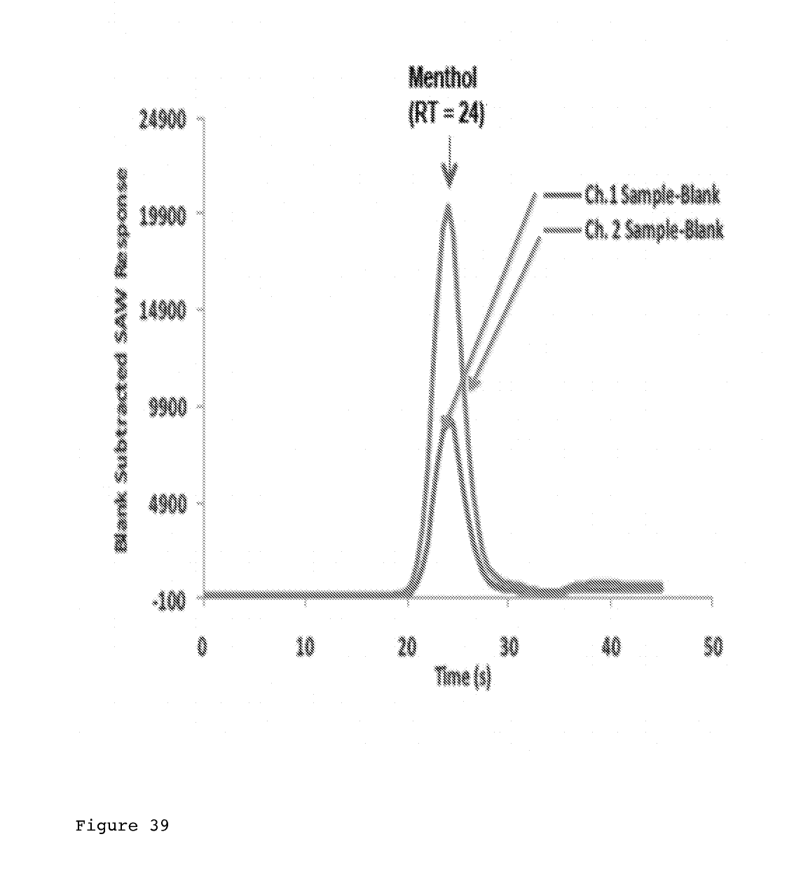

FIG. 39: SAW Reference Standards. Menthol headspace sample. Using the current configuration, D-limonene and menthol co-elute and show a similar relative response between the two detectors.

### Section 3: Qualitative SAW Analysis of Breath Samples Following Oral Administration of Alavert™ Tablets and FONA Wintergreen Flavoring Powder.

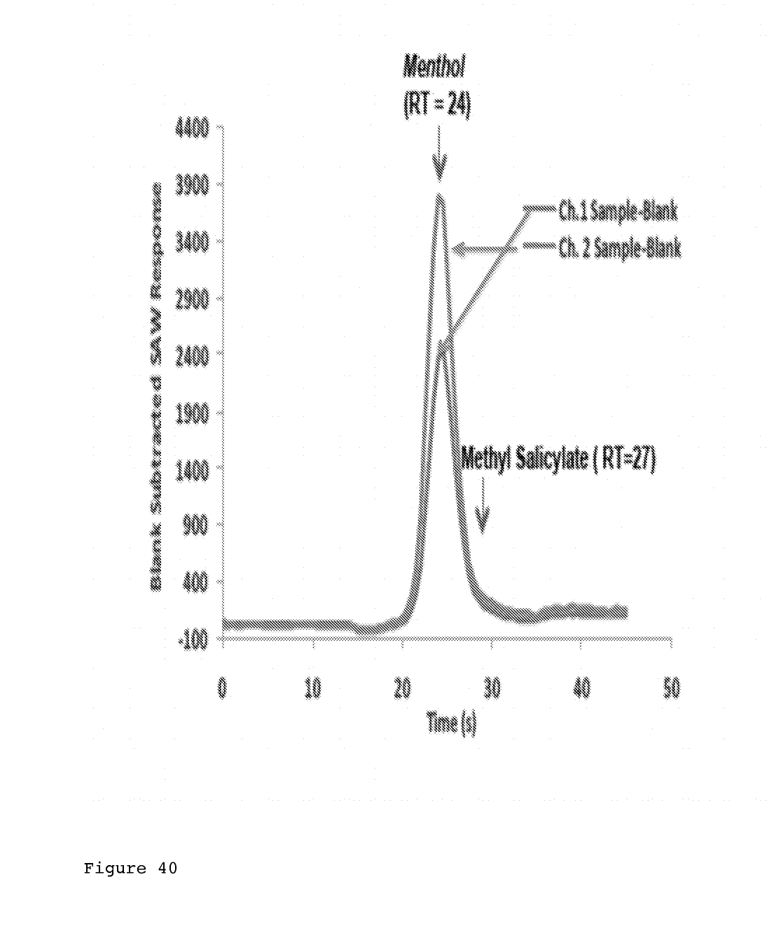

FIG. 40: Alayert™ Fresh Mint ODT. The chromatogram shown was obtained following the administration of a single Alayert™ Fresh Mint ODT tablet. The tablet was allowed to dissolve for in the mouth for 25 before the test subject blew a single breath sample into the SAW unit. Given the results of the GCMS analysis, this component is most likely menthol.

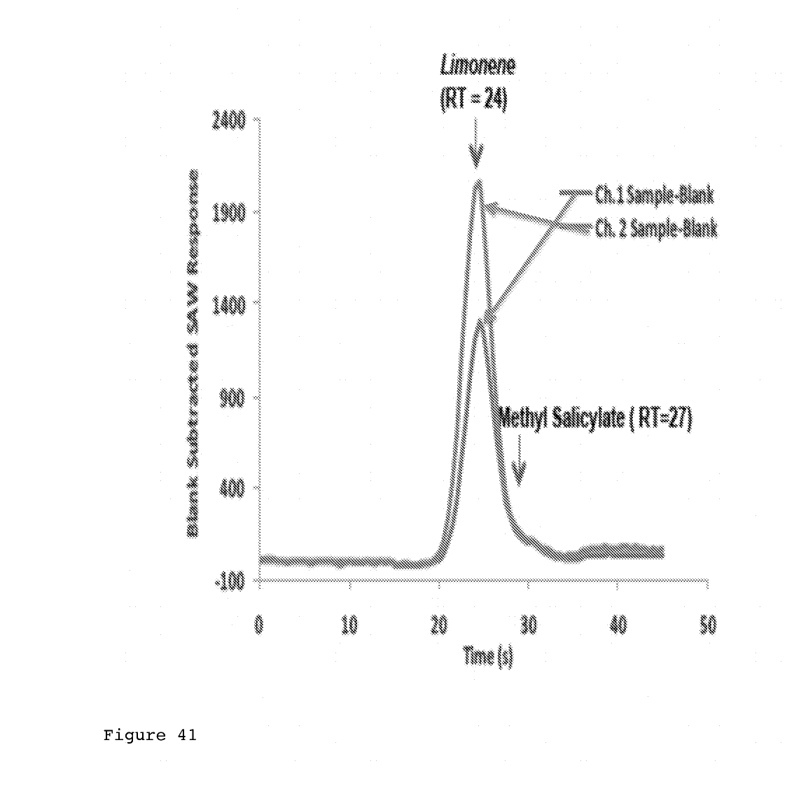

FIG. 41: Alayert™ Citrus Burst ODT. The chromatogram shown was obtained following the administration of a single Alavert™ Citrus Burst ODT tablet. The tablet was allowed to dissolve in the mouth for 25 s before the test subject blew a single breath sample into the SAW unit. Given the results of the GCMS analysis, this component is most likely D-limonene.

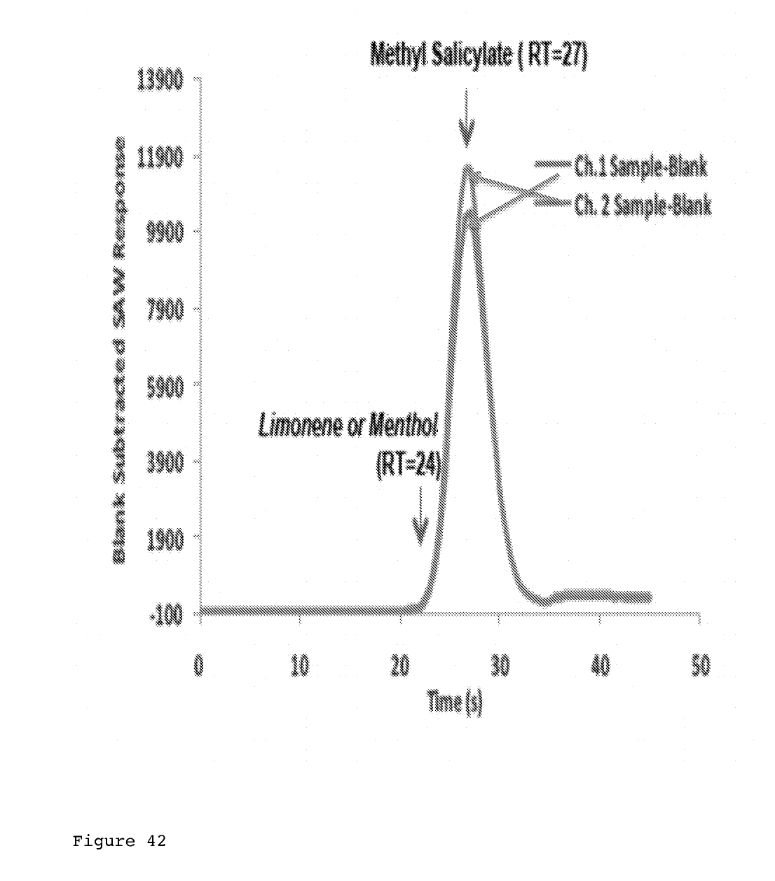

FIG. 42: FONA Wintergreen Powder. The chromatogram shown was obtained following the administration of 10 mg of the FONA Wintergreen powder. The powder was allowed to dissolve in the mouth for 25 s before the test subject blew a single breath sample into the SAW unit. Given the results of the GCMS analysis, this component is most likely methyl salicylate. The amount of methyl salicylate contained in 10 mg of the FONA powder produced a breath signal that was −40 times the response of a 100 ng standard. Less than 1 mg of this powder should be detectable in a tablet.

What is claimed is:

1. A Solid Oral Dosage Form (SODF) comprising a marker composition and an Active Pharmaceutical Ingredient (API) wherein said marker composition and said API are not in direct contact with each other, unless said marker is known to be compatible with said API, wherein said marker composition comprises at least one directly detectable Exhaled Drug Ingestion Marker (EDIM), or at least one marker which is metabolically converted into an EDIM, or combinations thereof.

2. The SODF according to claim 1 wherein said SODF comprises either (a) a tablet comprising said API, (b) a capsule comprising said API, or (c) particles containing said API.

3. The SODF according to claim 2 wherein, in addition, said SODF comprises said marker composition in a format selected from the group consisting of: (a) a tablet (b) a coating surrounding said API (c) a capsule (d) loose particles (e) particles contained within a tablet (f) particles contained within a capsule (g) particles surrounding said API wherein said particles and said API are contained within a capsule which contains both and (h) combinations thereof.

4. The SODF according to claim 3 wherein said SODF has a form selected from any of the forms shown in FIG. 2 or 3.

5. The SODF according to claim 1 wherein said marker comprises at least one flavorant which gives rise to an Exhaled Drug Ingestion Marker (EDIM) if the SODF is an Orally Disintegrating Tablet, (ODT), sublingual tablet, chewable tablet or, for other types of SODFs, said marker comprises at least one secondary or tertiary alcohol, at least one ketone, or both, for definitive medication adherence monitoring, wherein said at least one secondary or tertiary alcohol and said at least one ketone are each non-toxic at the dosage included in said SODF, wherein said ketone or tertiary alcohol is directly detectable in exhaled breath of a subject as an Exhaled Drug Ingestion Marker (EDIM) and wherein said secondary alcohol is detectable as an EDIM following metabolism to a ketone metabolite of said alcohol.

6. The SODF according to claim 5 wherein said secondary alcohol is selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-methyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol.

7. The SODF according to claim 5 wherein said ketone is the ketone of a secondary alcohol selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-methyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol.

8. The SODF according to claim 5 wherein said SODF is an ODT and said marker is selected from the group consisting of ethyl vanillin, vanillin, benzaldehyde, cinnamaldeyde, methyl anthranilate, methyl salicylate, DL-menthol, menthone, D-limonene, L-carvone, or combinations thereof.

9. The SODF according to claim 1 wherein several hard tablets, capsules, or APIs are packaged into a single SODF.

10. The SODF according to claim 9 wherein for each said hard tablet, capsule or API, there is provided a unique marker composition.

11. The SODF according to claim 10 wherein each said unique marker composition comprises at least one directly detectable EDIM, or a marker which is detectable as an EDIM upon metabolic activity which produces an EDIM from said marker, or both.

12. A method for monitoring subject adherence with a medication regimen which comprises (i) providing to said subject a Solid Oral Dosage Form (SODF) comprising a marker composition and an Active Pharmaceutical Ingredient (API) wherein said marker composition and said API are not in direct contact with each other and said marker is either directly detectable in exhaled breath of a subject as an Exhaled Drug Ingestion Marker (EDIM) or which is metabolically converted into an EDIM that is detectable in exhaled breath of a subject, or both; and (ii) monitoring the exhaled breath of said subject to detect said directly detectable EDIM or said metabolically produced EDIM or both.

13. The method according to claim 12 wherein said SODF comprises either (a) a tablet comprising said API, (b) a capsule comprising said API, or (c) particles containing said API.

14. The method according to claim 13 wherein, in addition, said SODF comprises said marker composition in a format selected from the group consisting of: (a) a tablet (b) a coating surrounding said API (c) a capsule (d) loose particles (e) particles contained within a tablet (f) particles contained within a capsule (g) particles surrounding said API wherein said particles and said API are contained within a capsule which contains both and (h) combinations thereof.

15. The method according to claim 12 wherein said SODF has a form selected from any of the forms shown in FIG. 2 or 3.

16. The method according to claim 12, wherein said marker composition comprises either a flavorant which gives rise to an Exhaled Drug Ingestion Marker (EDIM) if the SODF is an Orally Disintegrating Tablet, (ODT) or, if not an ODT, said marker comprises at least one secondary or tertiary alcohol, at least one ketone, or both for definitive medication adherence monitoring, wherein said secondary alcohol(s) and said ketone(s) are each non-toxic at the dosage included in said SODF, wherein said ketone or tertiary alcohol is directly detectable in exhaled breath of a subject as an Exhaled Drug Ingestion Marker (EDIM) and wherein said secondary alcohol is detectable as an EDIM following metabolism to a ketone metabolite of said alcohol.

17. The method according to claim 16 wherein said secondary alcohol is selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-methyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol.

19. The method according to claim 16 wherein said ketone is the ketone of a secondary alcohol selected from the group consisting of 2-propanol, 2-butanol, 2-pentanol, 3-pentanol, 3-methyl-2-butanol, 3-hexanol, 2-hexanol, 3-methyl-2-pentanol, 4-methyl-2-pentanol, 2,4-dimethyl-3-pentanol, 3-methyl-3-hexanol, 2,6-dimethyl-4-heptanol, 2-heptanol, 3-heptanol, 4-heptanol, 5-methyl-3-heptanol, 6-methyl-3-heptanol, cyclopentanol, cyclohexanol, 4-isopropylcyclohexanol, and trimethylcyclohexanol.

20. The method according to claim 16 wherein said SODF is an ODT and said marker is selected from the group consisting of vanillin, benzaldehyde, methyl anthranilate, methyl salicylate, DL-menthol, D-limonene, L-carvone, or combinations thereof.

21. The method according to claim 12 wherein several hard tablets, capsules, or APIs are packaged into a single SODF.

22. The method according to claim 20 wherein for each said hard tablet, capsule or API, there is provided a unique marker composition.

23. The method according to claim 21 wherein each said unique marker composition comprises at least one directly detectable EDIM and/or at least one marker which is detectable as an EDIM upon metabolic activity which produces an EDIM from said marker.

24. The method according to claim 22 wherein the ratio of said directly detectable EDIM to said EDIM produced upon metabolic activity is monitored.
